(19) United States
(12) Patent Application Publication
(10) Pub. No.: US 2007/0207469 A1
(43) Pub. Date: Sep. 6, 2007

(54) Reactive mixture with growing molecular species

(76) Inventor: Liang Wang, Acworth, GA (US)

Correspondence Address: LIANG WANG, 2935 WHITEVIEW DRIVE, NW, ACWORTH, GA, 30101, US

(21) Appl. No.: 11/368,132

(22) Filed: Mar. 3, 2006

(51) Int. Cl. C12Q 1/68; B67D 5/00; C12M 3/00

(52) U.S. Cl. 435/6; 427/2.11; 435/287.2; 222/81

(57) ABSTRACT

This invention relates to a method for preparing and dispensing a reactive mixture including at least a growing molecular species formed by mixing multiple reactive components in a pressure dispenser with the features of precise ratio, complete mixing, impact-activation, safeguard, and self-contained package.

### REFERENCE CITED

U.S. Patent Document

| | | | |
|---|---|---|---|
| 3,251,420 | May 17, 1966 | Rodgers, et al. | 169/77 |
| 3,591,089 | Jul. 6, 1971 | Cronan, | 239/304 |
| 3,718,235 | Feb. 27, 1973 | Cronan | 222/145 |
| 3,773,264 | Nov. 20, 1973 | Cronan | 239/304 |
| 3,993,224 | Nov. 23, 1976 | Harrison | 222/135 |
| 4,121,772 | Oct. 24, 1978 | Cronan | 239/304 |
| 4,377,256 | Mar. 22, 1983 | Commette | 239/117 |
| 4,469,252 | Sep. 4, 1984 | Obrist | 222/135 |
| 4,593,836 | Jul. 10, 1986 | Lilienthal | 222/136 |
| 4,666,062 | May 19, 1987 | Pershall | 222/136 |
| 4,801,046 | Jan. 31, 1989 | Miczka | 222/136 |
| 4,808,006 | Feb. 28, 1989 | Kaufeler | 222/136 |
| 4,969,579 | Nov. 13, 1990 | Behar | 222/136 |
| 4,979,638 | Dec. 25, 1990 | Bolduc | 222/1 |
| 5,018,643 | May 28, 1991 | Bolduc | 222/1 |
| 5,052,585 | Oct. 1, 1998 | Bolduc | 222/1 |
| 5,064,121 | Nov. 12, 1991 | Bolduc | 239/309 |
| 5,167,347 | Dec. 1, 1992 | Wiegner, et al. | 222/136 |
| 5,242,115 | Sep. 7, 1993 | Brown | 239/414 |
| 5,332,125 | Jul. 25, 1994 | Schmitkons, et al. | 222/1 |
| 5,344,051 | Sep. 6, 1994 | Brown | 222/135 |
| 5,405,051 | Apr. 11, 1995 | Miskell | 222/23 |
| 5,439,137 | Aug. 8, 1995 | Grollier et al. | 222/1 |
| 5,456,386 | Oct. 10, 1995 | Jesswein | 222/136 |
| 5,529,245 | Jun. 25, 1996 | Brown | 239/390 |
| 5,499,745 | Mar. 19, 1996 | Derin, at al. | 222/136 |
| 5,638,992 | Jun. 17, 1997 | Lim et al. | 222/129 |
| 6,283,221 | Sep. 4, 2001 | Hurray et al. | 169/30 |
| 6,513,729 | Feb. 4, 2003 | Ochiai, et al. | 222/135 |
| 6,520,377 | Feb. 18, 2003 | Yquel | 222/1 |
| 6,660,325 | Dec. 9, 2003 | Holfter, et al. | 427/121 |
| 6,691,898. | Feb. 17, 2004 | Hurray et al. | 222/190 |
| 6,755,348 | Jun. 29, 2004 | Langeman | 239/10 |
| 6,848,601 | Feb. 1, 2005 | Greer, Jr | 222/136 |

### OTHER REFERENCES

Studies of Surface Science and Catalysis, Vol. 131, By Dragutan, V. and Streck, R., Catalytic Polymerization of Cycloolefins, Elsevier Science B.V, 2000, Netherlands

### BACKGROUND OF THE INVENTION

1. Field of Invention

This invention relates to a method for preparing and dispensing a reactive mixture including at least a growing molecule species from multiple reactive components in a pressure dispenser under ambient temperature with the features of precise ratios, complete mixing, impact-activation, transportation safeguard, and self-contained package. The pressure dispenser comprises a main chamber loaded with the first reactive component and at least an accessory chamber loaded with the second reactive component. Both reactive components are prepared with precise component ratio following an optimum formulation. When accessory chamber is imploded by impacts when dispenser is under vigorous shaking, the reactive components are complete mixed and reacted to form reactive mixture under additional shaking. During the pot life when the nozzle is depressed causing valve to open under propellant pressure, the content is dispensed or sprayed.

2. Cross-Reference to Related Applications

A method for storing and dispensing a final product with pressure dispenser is known art. However, it is unknown that a method for preparing and dispensing a reactive mixture including at least a growing molecule species with pressure dispenser having features of precise ratio, complete mixing, impact-activation, safeguard, and self-contained package

3. Description of the Related Art

The dispenser including an aerosol can for dispensing a final product is a well-known art. However, a method for preparing and dispensing a reactive mixture including growing molecule species with pressure dispenser having the features of precise ratios, complete mixing, impact-activation, safeguard, and self-contained package is unknown.

Many processes require the application of reactive mixtures instead final products. For example, a reactive mixture of epoxy resin and amine hardener is prepared for application as an adhesive, and the final product is in a cross-linked solid state product and cannot be applied.

The methods or dispenser devices are known art that are holding plural products in separate containers and are being admixed as they are being discharged. Those “dynamic and continuous flow” methods or devices represent one group of previous arts, such as U.S. Pat. No. 3,993,224, U.S. Pat. No. 4,377,256, U.S. Pat. No. 4,469, 252, U.S. Pat. No. 4,801,046, U.S. Pat. No. 4,969,579, U.S. Pat. No. 5,242,115, U.S. Pat. No. 5,270,013, U.S. Pat. No. 5,332,125, US Pat. No. 5,344,051, U.S. Pat. No. 5,529,245, U.S. Pat. No. 5,499,745, U.S. Pat. No. 6,283,221, U.S. Pat. No. 6,513,729, U.S. Pat. No. 6,520,377, U.S. Pat. No. 6,660,325, U.S. Pat. No. 6,691,898 and U.S. Pat. No. 6,755,348.

A precise ratio of reactants is required for complete or total consumption of reactive groups or reactive sites of reactants, based on an equivalent functionally, such as the a ratio for an adhesive made of epoxy-amine, or a paint made of polyol-urethane during cross-linking step. In most cases various catalysts are added for acceleration the reaction rate. In an industrial practice, an optimum component ratio for reactive components to be consumed that is required to form an optimum final product by reactions to achieve the best physical properties for application.

The flow of liquids is viscosity dependent. Viscosity of liquids depends on concentrations of compositions, temperature, velocity of flow, the surface properties of the wall, etc. Industrial interested liquids usually contain multi-components, for example, high molecular weight polymer, pigment solids, colloids, etc. The flow behaviors of liquid media with different compositions are quite different. Therefore, it is very difficult even to control an approximate ratio of two components with a simple “dynamic and continuous flow” device. However, the optimum component ratio for multi-component mixture is a key requirement. Therefore, a complicated precise metering, ratio controlling, monitoring, and compensating system is required to achieve dispensing of multiple component mixture with a “dynamic and continuous flow” device. It may practical and economical only for big scale, ratio closing to 1:1, and repeat applications of few fixed combinations of mixtures with experienced workers. However, “dynamic and continuous flow” methods or devices are not suitable with small touchup jobs due to a lack of precision or too complicated for satisfy a precise component ratio requirement.

A common drawback of “dynamic and continuous flow” method involving solid product or an intermediate is the clog problem in the parts where mixed multi-components are passing. A reactive mixture involving solid product or intermediate will adhere to any parts, such as walls, cores, and tubes that contacted with the reactive mixture. For avoid blockage of passage, it is important practice to cleaning all parts of such “dynamic and continuous flow” devices after each application. It is time consuming and costly.

Other kind of previous arts represent the group with “seal-opening and batch mixing” U.S. Pat. No. 3,251,420, U.S. Pat. No. 3,591,089, U.S. Pat. No. 3,718,235, U.S. Pat. No. 3,773,264, U.S. Pat. No. 4,121,772, U.S. Pat. No. 4,593,836, U.S. Pat. No. 4,808,006, U.S. Pat. No. 4,979,638, U.S. Pat. No. 5,018,643, U.S. Pat. No. 5,052,585 U.S. Pat. No. 5,064,121, U.S. Pat. No. 5,405,051, U.S. Pat. No. 5,439,137, U.S. Pat. No. 5,456,386, and U.S. Pat. No. 5,638,992 represent such “seal broken & batch mixing” devices. However, all those arts are lake of mixing completeness. For satisfy a precise component ratios, all reactive components need to be released into a mixing chamber without residue concentration gradient. In all those inventions, when the seal is opening, the component originally stored in the compartment or container only can be partially released to the mixing chamber. The opened or broken chamber is still hold a part of component on the wall or in the space where complete mixing is impossible due to blocked geometry.

U.S. Pat. No. 3,251,420, U.S. Pat. No. 3,591,089, U.S. Pat. No. 3,718,235 and U.S. Pat. No. 3,773,262 have a common drawback that is lack of seal hermetization of the inner container for second component, lack of safeguard for transportation and accidental collisions, and redundant non-functional parts.

U.S. Pat. No. 4,121,772 has the drawback of complicated and redundant cylindrical cartridge member and an integral plastic member to mount the secondary container. The lack of seal hermetization is a major problem.

U.S. Pat. No. 4,593,836 used a displaceable plug for actuate “open seal”. However, sealing surrounding the removable plug is difficult due to where the sealing element is always contacting with liquid phase propellant and solvents. For reducing the leaking of contents, sealing material must made of expensive perfluorinated elastomer.

U.S. Pat. No. 4,666,062 and U.S. Pat. No. 5,167,347 is lack of completion of release and mixing of two components.

U.S. Pat. No. 4,808,006 is lack of seal hermetization of movable rod. The drawback is lack of completely release of second component.

U.S. Pat. No. 5,405,051 used a piercing tube which has same drawback due to sealing requirement as U.S. Pat. No. 4,693,836.

U.S. Pat. No. 5,456,386 is lack of safeguard for collisions. The inner container may be separated from the sealing position by an accidental collision. Other drawback is the lack of completion of mixing of two components.

U.S. Pat. No. 4,979,638, U.S. Pat. No. 5,018,643, U.S. Pat. No. 5,052,585 and U.S. Pat. No. 5,064,121 used transparent external containers. Glass is fragile. Transparent plastic is fragile, low mechanical strength, and liable by attacked with propellants and organic solvents. Glass and plastics are not suitable for pressured containers for external package. Other common drawbacks in those inventions are push rods which were used to break ampoule, and are difficult being sealed under pressure.

U.S. Pat. No. 5,439,137 and U.S. Pat. No. 5,638,992 use the pressure releasing in outside chamber to produce a positive pressure in inner chamber and than to “open the seal”. The drawback in those devices is that substantial propellant and materials would be lost before the “open seal” could occur. The amount of released material required is multi parameter dependent variable, such as temperature, composition molar ratio, partial pressure of each component. To keep a precise ratio of two components become impossible.

U.S. Pat. No. 6,848,601 used two containers with a coupler to mix two components. It is not self-contained under the means for each container. It has safeguard advantage. This design has a disadvantage of difficult to operation by un-trained user, and double the manufacture cost compare to single device. It is only justified for high risk products that require 100% guarantee of no admixing of two components, such as two component explosive for deactivation of mines for professionals.

4. Objects and Advantages

Accordingly an object of this invention is to provide a storing means for plural reactive components prior to mixing in a pressure dispenser as a reservoir by storing plural reactive components with precise optimum component ratios loaded in separate compartments and maintained separately from one another until ready for use.

Another object of this invention is to provide a complete mixing means by a easily activation method and achieve a complete release and mixing of reactive components in the pressure dispenser implement an activation of releasing, mixing, and reacting plural reactive components originally stored in separate compartments prior to use.

Another object of this invention is to provide a preparing means of reactive mixture with complete release and mixing of reactive components in the pressure dispenser as a mixer and reactor for mixing and reacting plural reactive components originally stored in separate compartments prior to use.

A further object of the invention is to provide a dispensing means for reactive mixture with optimum component ratios containing growing molecule species prepared in pressure dispenser as dispenser means to discharge reactive mixture with easy operation.

A still further object of the invention is to provide an aerosol spraying means for reactive mixture prepared in pressure dispenser as aerosol spraying means to spraying reactive mixture with optimum component ratios containing growing molecular species.

A still further object of the invention is to provide a safeguard means for pressure dispenser as a dispenser for preparing and dispensing reactive mixture with optimum component ratios containing growing molecule species to avoid non-intention activation of admixing of reactive components originally stored in separate compartments during the transportation or accidental collisions.

5. The Advantages of This Invention Are:

(1) To provide a method for preparing and dispensing reactive mixture including at least a growing molecule species.

(2) To provide total release and complete mixing of multi reactive components by an inherent advantage of this invention with an implosion of accessory container where the accessory reactive component is originally stored. Following implosion, the wall of the accessory container is totally shattered, therefore, the content of all reactive components are completely mixed. The implosion of accessory container is safe for external wall of pressure container. In the opposite, explosion is danger due to the possibility of broken the external wall of the pressure container.

(3) To provide reliable means to allow reactive components and propellant with precise measured amounts to be loaded in separate plural compartments and containers.

(4) To provide reliable reservoirs for storing separately the reactive components in different compartments prior to mixing of multi reactive components.

(5) To provide easy means for dispensing and spraying reactive mixture with optimum component ratios containing growing molecule species formed in the dispenser under the propellant pressure.

(6) To provide a safeguard for the compartments and containers loaded with reactive components to avoid accidental activation of admixing of reactive components during transportation and accidental collisions.

(7) To providing an easy means for activation the implosion of the accessory container to trig the mixing and reacting of the multi reactive components.

(8) To integrate all functions, preparing and dispensing in one self-contained package and in portable, hand hold pressure dispenser.

(9) To provide economical manufacture of portable, independent devices for this invention.

### BRIEF SUMMARY OF THE INVENTION

These and other objects and advantages are achieved with present invention that relates to a method for preparing and dispensing a reactive mixture including at least a growing molecule species formed by mixing and reacting multiple reactive components with precise component ratios stored originally in separate chambers of a pressure dispenser with the features of precise ratio, complete mixing, impact-activation, safeguard, and self-contained package.

This invention relates to a method for preparation and dispensing a reactive mixture containing at least a growing molecule species under ambient temperature in a pressure dispenser comprising a main chamber loaded with the first reactive component and at least an accessory container loaded with the secondary reactive component with an optimum component ratio of reactants, catalyst, additives, and other inertia ingredients required by formulation. When the dispenser is under vigorous shaking intentionally, the accessory container is imploded by impacts; the reactive components are completely mixed and reacted to form reactive mixture containing growing molecular species with optimum component ratios under additional shaking. Pre-filled liquefied propellant provides a pressure source in the dispenser. The propellant is dissolved in the reactive mixture with optimum component ratios containing at least a growing molecule species. When the nozzle is depressed causing valve to open under propellant pressure, the content is dispensed. A safeguard design is integrated to avoid the impact activation during transportations or by unintentional accidental collision.

### BRIFE DESCRIPTION OF DRAWING

FIG. 1, FIG. 2, and FIG. 3 show the method of preparation and dispersion of a reactive mixture containing growing molecule species with a pressure dispenser of the present invention.

FIG. 1 illustrates the method by a sectional view of reactive components prepared in precise component ratio and stored in main chamber and accessory container of a pressure dispenser prior to mix and the safeguard feature.

FIG. 2 illustrates the method by a sectional view of complete mixing of reactive components by implosion of the accessory container with vigorous shaking of the pressure dispenser during preparation of the reactive mixture.

FIG. 3 illustrates the method by a sectional view of dispensing reactive mixture containing growing molecular species with the pressure dispenser.

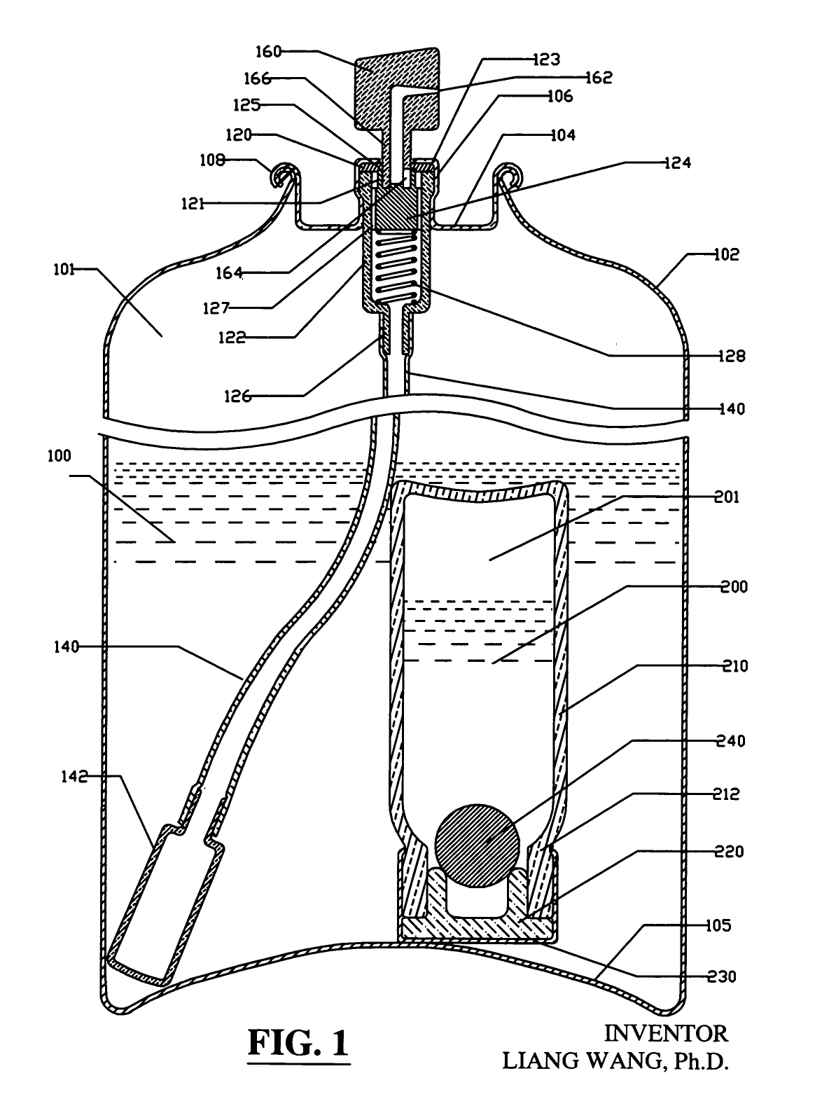

Referring to FIGS. 1, 2 and 3, there is shown the preparation and dispensing a reactive mixture containing at least a growing molecule species by pressure dispenser 102 of present invention.

Referring to FIG. 1, a mixture of the first reactive component and liquefied propellant loaded in the main chamber of pressure dispenser 102 has liquid phase 100, and vapor phase 101, and the second reactive component loaded in accessory chamber, the glass vial 210, has liquid phase 200 and vapor phase 201. Each of reactive components is loaded with precise ratios according to optimum ratios and maintained separately from one another within the main chamber of pressure dispenser 102, and the accessory chamber 210 with a safeguard feature of 220 which prevents the mixing of the said reactive components until activation.

The separation between reactive components (100, 101 ) and (200, 201) in pressure dispenser 102 therein guarantees substantial shelf life since there is no reaction between reactive components (100, 101) and (200, 201) within the pressure dispenser 102 prior to the mixing of the materials.

The reactive mixture 300 containing at least a growing molecule species within pressure dispenser 102 is prepared by vigorous shaking that results the implosion of accessory chamber 210 and mixing of reactive component (100, 101) and (200, 201 ) immediately prior to use so that at least a growing molecule species is active and the increase of viscosity of reactive mixture is tolerable for dispensing.

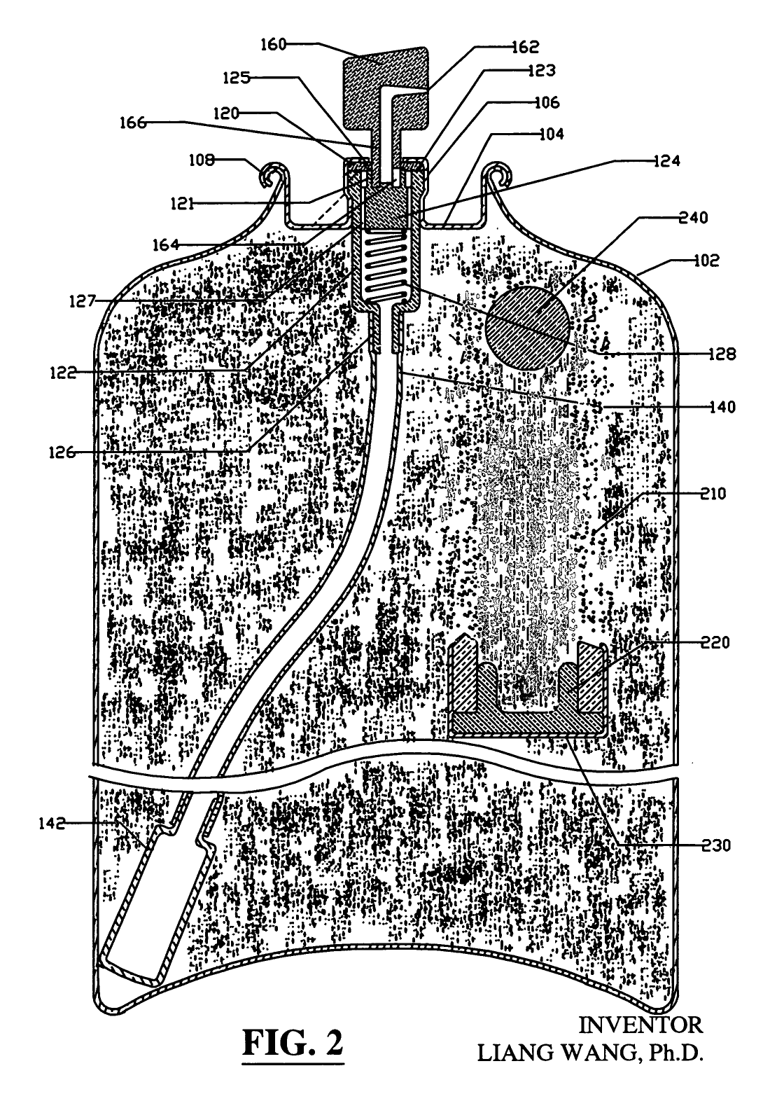

Referring to FIG. 2, the mixing of reactive components is induced by the implosion of said accessory container upon impact with a core-element 240 included in said accessory container with vigorous shaking.

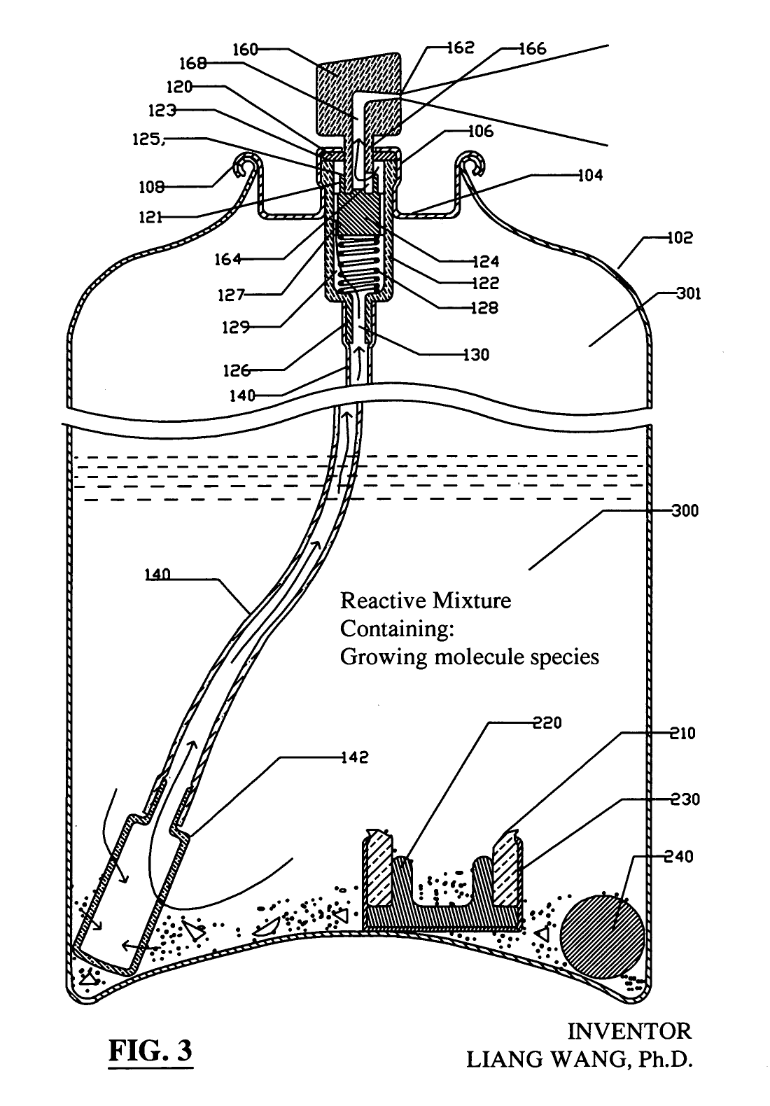

Referring to FIG. 3, the deliver of the mixture 300 of reactive mixture containing growing molecule species and the propellant to a desired location is shown. A combined fill and discharge valve 124 mounted in the valve housing 122 which is sealed with resilient gasket 120 underneath center opening of pedestal 106. When the discharge is actuated the product under propellant pressure is dispensed through the slide valve core 124, and finally, exiting through the dispenser spout 162.

### DETAILED DESCRIPTION OF THE INVENTION

A broad range of consumer products use dispensers as self-contained package. Those products include one component paints, foams, adhesives, lubricants, insecticides, repellents, foodstuffs, cosmetics, skin-care, hair styles, cleaners, deodorizers, medicine inhaler, personal-defending, tracers, and so on. A typical dispenser is a cylindrical can. A single component product is stored in the can filled with a pressured gas or a liquefied gas. The dispenser has a passage equipped with a shut-off valve connected with a nozzle. When the valve is actuated the product under pressure is dispensed through the valve and exited from the nozzle.

Many applications, however, such as multi component ambient temperature cured paints, adhesives, and foams do not use final products, but apply reactive mixtures with optimum component ratios for achieve an optimum final product. The final products of multi-component coatings, foams, and adhesives are in solid state; therefore, it is impossible to apply.

A self-contained package method for preparing and dispensing reactive mixture with optimum component ratios of multi-components is not commercialized. The inherent problem is the complexity of chemical reactions and reaction kinetics involved. For example, curing reactions in the chemical sense requires a ratio of reactants for total consumption of reactive groups or reactive sites, based on an equivalent functionality, such as for the paint of polyol-urethane, the adhesive of epoxy-amine during hardening step. Catalysts are required to accelerate chemical reactions under ambient temperature at desired reaction rate. The ratio of the amount of catalyst to the specific reactive components is also critical. The optimum component ratios including the ratio of catalyst, and additives to reactive components are required to form a final product with optimum physical properties.

Thousands of brands of polymers, oligomers, cross-linkers, catalysts, and additives are used in industry. All have very different properties, such as the molecular weights, functional group values, and viscosities. For example epoxy resin can react and cure with well over 50 different classes of chemicals, such as amines, polyamides, anhydrides, Lewis acids, ureas, melamine, imidazoles, BF, amine complexes, imides, and so forth. All curing agent has different functionality and require different ratio to reach optimum cure. In addition, catalysts are required for acceleration the reactions for a desired reaction rate. To achieve a desired reaction speed, a precise ratio of specific catalyst to special reactive components is also required. There is no convenient self-contained package and simple dispensing method that can meet the requirements associated for preparing reactive mixture with precise multi-component reactant ratios.

A growing molecule species is a living molecule with active functional group and with feature of the increase of molecule weight in the progress of the time. In the course of time it will convert into a more stable molecule after active function group reacted. A growing molecule species occur typically during polymerization, oligomerization, cross-linking, colloid gelation, sol-gel networking, and cluster growing reactions. The molecule with functional group or groups, such as monomer, oligomer, and polymer, are growing during chemical reaction. A growing molecule species differs from reactants and products in the way that it is an intermediate molecule that only exists in chemical reaction stages.

During a cross-linking reaction a growing molecule species has the character that molecule weight is increasing and bond network is expending and cross-linking. During cross-link reactions a reactive mixture containing molecular species with growing molecular weight is formed. For example, a multi-component reactive mixture containing molecule species with growing molecular weight of polyolurethane-catalyst complex is prepared by mixing polyol, urethane and catalyst.

After mixing, hydroxyl groups in polyol are reacting with isocyanato groups in urethane to form urethane bonds under the interacting of metal complex catalyst. A partially reacted polyol molecular has free hydroxyl groups coordinated with metal complex and has sites with cross-linked urethane bonds resulting in the reactions with isocyanato groups. They are chemically reactive. The molecular weights of them are continuously growing. Before all hydroxyl bonds are exhausted, the chemically reactive molecule species are continuously growing, and chemical bond networks are expending until the formation of final cross-linked product.

In chemical sense a stoichiometric ratio of reactants is required for complete or total consumption of reactive groups or reactive sites. In practice, for example, the water molecules exist in other raw materials such as in pigments and in solvents, on the surfaces to be coated and in environmental that also consumes the isocyanato groups of urethane reactant. The ratio of urethane reactant should be higher than the stoichiometric ratio to compensate extra consumption. The precise ratios for reactive components to be consumed are required for an optimum final product with the achievable optimum physical properties. This precise ratio is called optimum component ratio for particular reactant components and determined by experiments.

For forming optimum polyurethane final product an optimum component ratio of polyol/urethane and polyol/catalyst are required. During the preparation of multi component polyurethane paint the reactive mixture with optimum component ratios containing growing molecule species is provided by mixing and reacting polyol, urethane, and metal complex catalyst with precise ratios according to an optimum formulation.

After mixing, a reaction usually undergoes an inductive period with low reaction rate, and then the reaction rate increase progressively. Following the progress of mutual reactions of the functional groups, forming of cross-linked chemical bonds, and growing of molecular weights, the flow viscosity is increasing dramatically. The structural transition occurs from liquid to a gelation and vitrification; and finally becomes solid state which cannot be dissolved or melted. A pot life defines a time span that the mixture is able to flow freely after mixing. During the pot life the reactive mixture remains low viscosity, and can be dispensed under propellant pressure. The pot life is limited typically within several minutes to several hours. Therefore, it requires maintaining those multiple reactive components in separate containers until desired to admixing. Such reactive components have typically been packaged in separate containers and mixed when just prior to use. During a preparation, reactants, catalysts, pigments, additives, and reactive diluents are precisely weighted or measured separately. The solvents are precisely measured for reaching a desired flow viscosity. Multiple reactive components, catalysts, inertia components, additives, and solvents are mixed thoroughly to forming the reactive mixture ready for applying. Accordingly, they have generally not been suitable for use in the more convenient self-contained package, such as pressure dispensing containers.

Therefore, the main subject of the present invention is a method for preparing and dispensing a reactive mixture with optimum component ratios containing growing molecule species in a pressure dispenser for dispensing or spraying application. More specifically, the invention relates to a method for preparing and dispensing reactive mixture in a pressure dispenser which allows multiple reactive components to be loaded with optimum ratios and stored in separate compartments including at least one accessory container just prior to use. When the time desired for preparing the reactive mixture from multi reactive components with optimum component ratios, intentional vigorous shakings are performed. As result of the impacts, an implosion of the accessory container occurs, the accessory container is shattered, and the component originally stored in the accessory container is totally released into the main chamber and mixed with other reactive components. There is no geometric obstacle to interfere the complete mixing. After mixing and reacting of multi reactive components, the reactive mixture is prepared that can be dispensed under propellant pressure by actuating valve during the product pot life specified flow viscosity. A safeguard design is implemented for avoiding the unintentional impact activation during transportation or accidental collisions.

This invention relates to a method for preparation and dispensing a reactive mixture under ambient temperature in a pressure dispenser comprising a main chamber loaded with the first reactive component and at least an accessory container loaded with the secondary reactive component with an optimum component ratio of reactants, catalyst, additives, and other inertia ingredients required by formulation. When the accessory container is imploded by impacts, the reactive components are mixed and reacted to form an optimum reactive mixture containing growing molecule species. When the nozzle is depressed causing valve to open under propellant pressure, the content is dispensed. A safeguard design is integrated to avoid the impact activation during transportations or by unintentional accidental collision.

This invention relates generally for preparing and dispensing a reactive mixture with optimum component ratios containing growing molecule species in a pressure dispenser as dispenser device for dispensing the reactive mixture containing growing molecule species prepared by mixing and reacting multi reactive components with optimum ratios under ambient temperature, and including the spraying reactive mixture as aerosol generator. This invention is directed to an impact activation of the accessory container that is imploded when desired to initiate the mixing of multi reactive components with precisely prepared, loaded and stored separately in different chambers and containers in the pressurized dispenser. The dispenser is designed for holding at least two reactive components that cannot generally be mixed together until shortly before use More particularly, the mixing of multi reactive components is activated by impacts when the dispenser is under vigorously shaking by intention. When impacts are executed by intention by vigorous shaking, the accessory container is imploded resulting in the mixing of reactants, catalyst, additives, and other inertia ingredients loaded in multi compartment of the pressure dispenser. This invention provides a means for safeguarding the integrality of the accessory container during transportation to avoid undesired impact activation. This device provides a means for actuate the dispenser and discharging the reactive mixture for use under propellant pressure.

This invention relates to a method for preparation and dispensing of reactive mixture with optimum component ratios containing at least a growing molecule species in a pressure dispenser as a dispenser with the features of impact-activation and transportation safeguard for dispensing and spraying, such as for an aerosol application.

When the first reactive component contains a polyol macromolecule with at least two hydroxy functional groups and the second reactive component contains at least an isocyanate with at least two isocyanato functional groups, the reactions between those two reactive components form at least a growing molecule species containing at least a free hydroxy functional group.

When the first reactive component contains a polyamine macromolecule with at least two amino functional groups and the second reactive component contains at least an isocyanate with at least two isocyanato functional groups, the reactions between those two reactive components form at least a growing molecule species containing at least a free amino functional group

When the first reactive component contains an epoxy oligomer with at least two epoxy functional groups and the second reactive component contains at least a curing agent, said curing agent is selected from the group consisting of amine, polyamide, anhydride, Lewis acid, urea, melamine, imidazole, BF, amine complex, imide, and a mixture thereof. The reactions between those two reactive components form at least a growing molecule species containing at least a free epoxy functional group.

When the first reactive component contains an acrylic molecule with at least a carbon-carbon double bond and the second reactive component includes at least a compound selected from the group consisting of organic peroxide, inorganic peroxide, azo compound, metal alkyl, metathesis catalyst, Bronsted acid, Lewis acid, anionic catalyst, Zeigler-Natta coordination catalyst, organo-metallic compound, metal complex, and a mixture thereof. The reactions between those two reactive components form at least a growing molecule species containing at least a free carbon-carbon double bond.

When the first reactive component contains at least an cyclic olefin, said cyclic olefin is selected from the group consisting of monocyclic olefin, bicyclic olefin, polycyclic olefin, cyclic olefin with ester group, cyclic olefin with nitrile group, cyclic olefin with halogen group, oxygen-containing heterocyclic olefin, nitrogen-containing heterocyclic olefin, silicon-containing heterocyclic olefin and a mixture thereof, and said second reactive component includes at least a compound, said compound is selected from the group consisting of metathesis catalyst, Bronsted acid, Lewis acid, anionic catalyst, Zeigler-Natta coordination catalyst, organo-metallic compound, metal alkyl, metal complex, inorganic peroxide, organic peroxide, azo compound, and a mixture thereof. The reactions between those two reactive components form at least a growing molecule species containing at least a free carbon-carbon double bond.

When the first reactive component contains at least a polysulfide oligomer, and second reactive component includes at least a curing agent, said curing agent is selected from the group consisting of lead dioxide, activated manganese dioxide, calcium peroxide, cumene hydroperoxide, alkaline dichromate, p-quinonedioxime, furfurol, dichlorodiphenol, tine oxide, hydrazine, peperidine, magnesium oxide, sulfoxide, epoxy oligomer, isocyanate, potassium permanganate, zinc oxide, and a mixture thereof. The reactions between those two reactive components form at least a growing molecule species containing at least a free thio functional group.

When the first reactive component contains at least a fibrinogen, and said second reactive component includes at least collagen aggregation enzyme. The reactions between those two reactive components form at least a growing molecule species containing at least a free amino-acid functional group.

When the first reactive component contains at least a phenyldiamine, and second reactive component includes at least a dilute solution of hydrogen peroxide. The reactions between those two reactive components form at least a growing molecule species containing at least a free imino functional group.

When the first reactive component includes at least a dye certified for foods, drugs and cosmetics, said dye certified for foods, drugs and cosmetics is selected from the groups consisting of azo dye, diazo dye, cyanine dye, rhodamine dye, xanthere dye, fluorine dye, anthraquinone dye, triphenylmethane dye, indole dye, indoline dye, chromoionophore, fluoroionophore, melanin dye, and a mixture thereof, and second reactive component includes at least an agent with a functional group, said functional group is selected from the group consisting of thio, thioacetyl, thiobenzoyl, thiocarbamoyl, thiocarbazono, thiocarbodiazono, thiocarbonohydrazido, thiocabonyl, thiocarboxy, thiocyanato, thioformyl, thionoyl, thioreido, thioxo, mercapto, methionyl, acetylcysteine, cysteine, cysteino, cystine, cystino, cysteino, cystamino, epidithio, epithio, isothiocyanato, thioglycolate, thiolacetate, thioglycolate, thiolactate, thioacetyl, thiobenzoyl, thiocarbamoyl, thiocarbazonol, thiocarbodiazonol, and a mixture thereof. The reactions between those two reactive components form at least a growing molecule species containing at least an conjugated double bond chromophore functional group.

The invention further including a step following said dispensing reactive mixture including growing molecule species, said step is selected from the group consisting of heating, IR heating, microwave heating, UV irritation, electron beam irritation, grafting reaction, telomerisation reaction, telechelic reaction, and chemical modification.

The invention further including one or more steps following said dispensing reactive mixture including growing molecule species, said step is selected from the group consisting of heating, IR heating, microwave heating, UV irritation, electron beam irritation, grafting reaction, telomerisation reaction, telechelic reaction, and chemical modification.

The invention further including at least one step following said dispensing reactive mixture including growing molecule species, said step is selected from the group consisting of heating, IR heating, microwave heating, UV irritation, electron beam irritation, grafting reaction, telomerisation reaction, telechelic reaction, and chemical modification.

The dispenser is pressure resistant metal dispenser, equipped with an opening and is comprised of a main compartment being loaded with the first reactive component, and at least an accessory container being loaded with the second reactive component. Both the first reactive component and the second reactive component are precisely measured consisting with optimum component ratios. A vault metal top having valve housing is hermetically crimped and sealed in the opening of the dispenser. A normally closed shut-off valve connecting with a dispensing nozzle is mounted in the valve housing connecting to the main compartment and having a dipping tube ending with a filter to form a passage.

The second reactive component in the accessory container separates from first reactive component until the accessory container is imploded by impacts with core-element included in the accessory container during vigorous shaking. After the implosion the accessory container is shattered, the first reactive component and the second reactive component are mixed and reacted to form reactive mixture with optimum component ratios containing growing molecule species within the pressure dispenser. Pre-filled liquefied propellant provides a pressure source in the dispenser. The propellant is dissolved in the reactive mixture with optimum component ratios containing growing molecule species formed by the mixing and reacting of reactive components, the reactive mixture including growing molecule species and dissolved propellant is dispensed through a spout of the nozzle when the nozzle is depressed causing the valve to open.

This invention relates to a method for preparation and dispensing of reactive mixture with optimum component ratios containing growing molecule species in a pressure dispenser as a dispenser. Said pressure dispenser comprises a cylinder can made with metal having a concave bottom wall and a vault top that is crimpled on the cylinder. The dispenser is designed for holding at least two reactive components that are loaded with optimum component ratios and cannot generally be mixed together until shortly before use. The first reactive component is loaded in the main chamber, and the second reactive component is loaded in accessory container. A liquefied propellant is filled in the main chamber of the dispenser as pressure source. When impacts are executed by intention with vigorous shaking, the accessory container is imploded and shattered resulting in the complete mixing of reactants, catalyst, additives, and other inertia ingredients in the multi reactive components stored in the can. A reactive mixture with optimum component ratios containing growing molecule species is prepared following implosion of accessory container and mixing of multi reactive components when the dispenser is under vigorously shaking by intention. A valve connecting with a nozzle and having valve housing is mounted in the top that control dispensing. The housing has a dip tube dipping into the bottom of can. A filter is connected at the end of the dip tube. The dispensing valve held shut by the pressure inside of the can and a coil spring against a resilient gasket mounted in the valve housing. When the valve is actuated the reactive mixture containing growing molecule species under propellant pressure is dispensed through the valve and exited from the nozzle. When the mixture of reactive mixture with optimum component ratios containing growing molecule species and dissolved propellant is dispensed from a fine spout of the nozzle, the propellant evaporates as soon as the liquid drops of reactive mixture leave the spout and liquid drops are broken to fine mist aerosol.

These and other objects and advantages of the invention are achieved by using a metal dispenser as the first compartment or main chamber holding, under pressure, the first reactive component to be loaded with a precise amount for an optimum component ratios, and therein an accessory container is included in the main chamber as the second compartment holding the second reactive component to be loaded with a precise component ratios required by optimum formulation to form a reactive mixture. The accessory container as secondary compartment is imploded by multiple impacts by a core-element included inside the accessory container, when the dispenser is shaken vigorously by intention. In storage or during transportation, the integrity of said accessory container, the secondary compartment is maintained with a special design for safeguard the secondary compartment, and the reactive components are kept separate from one another. When it is desired to mix, said dispenser is shaken vigorously by intention, the accessory container is imploded and shattered by impacts with a core-element included inside said accessory container, and thereafter, the second reactive component is released into the main chamber and mixed and reacted with the first reactive component in the main chamber after said implosion and under vigorous shaking.

In one form of the invention, the accessory chamber is a glass vial having second reactive component with precise component ratios. The glass vial has an open end having a neck. The side wall of the glass vial is made thicker to resistant the accident fall with side collisions. The open end is plugged by a resilient plug. The resilient plug seals the second reactive component and the core-element. It provides resilient buffer against the impact force on the resilient plug by the core-element. The plug is caped on the neck with a thin metal cap. The metal cap holding on the vial neck restricts the plug on the face of vial open end.

In other form this invention is to provide a method for preparation and dispensing of reactive mixture with optimum component ratios containing growing molecule species in a pressure dispenser as a dispenser. Said pressure dispenser comprises a cylinder can made with metal having a concave bottom wall and a vault top that is crimpled on the cylinder. The dispenser is designed for holding at least three reactive components that are loaded with optimum component ratios and cannot generally be mixed together until shortly before use. The pressure dispenser comprises of a main compartment being loaded with the first reactive component, and at least two accessory containers, the first accessory container and the second accessory container, being loaded separately with the second reactive component and the third reactive component. All three reactive components are precisely measured and consist with optimum component ratios. In one form of the invention, the first accessory and secondary accessory containers are glass vials. A liquefied propellant is filled in the main compartment as pressure source.

When impacts are executed by intention with vigorous shaking, the accessory containers are imploded resulting from the impacts of core-elements originally inserted in the first accessory and second accessory containers. After implosion and shattering of the first and the second accessory containers, the mixing of all reactive components, including reactants, catalyst, additives, and other inertia ingredients in the multi reactive components stored in the pressure dispenser occurs under additional shaking. A reactive mixture with optimum component ratios containing growing molecule species is prepared following implosion and shatter of accessory containers and mixing of multi reactive components when the dispenser is under vigorously shaking by intention. A valve connecting with a nozzle and having valve housing is mounted in the top that control dispensing. The housing has a dip tube dipping into the bottom of can. A filter is connected at the end of the dip tube. The dispensing valve held shut by the pressure inside of the can and a coil spring against a resilient gasket mounted in the valve housing. When the valve is actuated by applied force with the nozzle depressed, a passage is opened and the reactive mixture containing reactive mixture with optimum component ratios containing growing molecule species is dispensed and exited from the nozzle.

In one typical form of the invention, the accessory containers are glass vials. Each glass vial has an open end having a neck. The side wall of the glass vial is made thicker to resistant the accident fall with side collisions. After a core-element, a reactive component are loaded, the open end is plugged by a resilient plug. The resilient plug seals the reactive component and the core-element. It provides resilient buffer against the impact force on the resilient plug by the core-element. The plug is caped on the neck with a thin metal cap. The metal cap holding on the vial neck restricts the plug on the face of vial open end.

The resilient plug is made of the elastomer that provides resilient property and resists the solvent and liquefied propellant. Solvent or propellant swollen should not occur. The solvent and propellant used should no effect on the plug under two year immersion.

The metal cap is made of metal or metal alloy that resists the chemical corrosions of the reactive components stored in the main chamber. When improved chemical resist is required, a cross-linked polymer film is coated on the surfaces of the metal cap for guarantee there is no corrosion or chemical reactions attacking the metal cap.

Under typical assembly of the vial, first, the core-element is inserted in the vial, second, a precisely measured volume of second reactive component is filled in the vial, the third, the resilient plug is plugged on the open end of vial, and finally, the plug is caped on the neck of the vial with a metal cap.

Under other assembly of the vial, first, the core-element is inserted in the vial, second, a precisely measured volume of second reactive component is filled in the vial, the third, the resilient plug is plugged on the open end of vial, the forth, the plug is caped on the neck of the vial with a metal cap, and finally, the vial is vacuumed by a inserted pipe dipping inside of vial.

Under other typical assembly of the vial, first, the core-element is inserted in the vial, second, the system is evacuated by vacuum, third, a precisely measured volume of second reactive component is filled in the vial under vacuum, the forth, the resilient plug is plugged on the open end of vial under vacuum, and finally, the plug is caped on the neck of the vial with a metal cap.

Other assembly procedure is practiced. First, the core-element is inserted in the vial, second, a precisely measured volume of second reactive component is filled in the vial, the third, the system is evacuated under vacuum, the forth, the resilient plug is plugged on the open end of vial under vacuum, and the fifth, the plug is caped with a metal cap on the neck of the vial.

A still other assembly procedure is practiced. First, the core-element is inserted in the vial, second, the resilient plug is plugged on the open end of vial, and third the plug is caped with a metal cap, and forth the vial is vacuumed by a inserted pipe dipping inside of vial, and finally, a precisely measured volume of second reactant component is filled in the vial.

In other form of the invention, the secondary compartment, the accessory container, having second reactive component and a core-element, is a glass vial having an open end having a screw-thread neck socket. The side wall of the glass vial is made thicker to resistant the accident fall with side collisions. The open end is plugged by a resilient plug. The plug is caped on the neck with a screw cap.

Under typical assembly of the vial, first, the core-element is inserted in the vial, second, a precisely measured volume of second reactive component is filled in the vial, the third, the resilient plug is plugged on the open end of vial, and finally, the plug is caped on the neck with a screw cap.

Under typical assembly of the vial, first, the core-element is inserted in the vial, second, a precisely measured volume of second reactive component is filled in the vial, the third, the resilient plug is plugged on the open end of vial, the forth, the plug is caped on the neck with a screw cap, and finally, the vial is vacuumed by a inserted pipe dipping inside of vial.

Under other typical assembly of the vial, first, the core-element is inserted in the vial, second, the system is evacuated under vacuum, the third, precisely measured volume of second reactive component is filled in the vial under vacuum, the forth, the resilient plug is plugged on the open end of vial under vacuum, and finally the plug is caped with a screw cap.

Other assembly procedure is practiced. First, the core-element is inserted in the vial, second, a precisely measured volume of second reactive component is filled in the vial, the third, the system is evacuated under vacuum, the forth, the resilient plug is plugged on the open end of vial under vacuum, and the fifth, the plug is caped with a screw cap.

Still an assembly procedure is practiced. First, the core-element is inserted in the vial, second, the resilient plug is plugged on the open end of vial, and third the plug is caped with a screw cap, and forth the vial is vacuumed by a inserted pipe dipping inside of vial, and finally, a precisely measured volume of second reactant component is filled in the vial.

The resilient plug seals the second reactive component and the core-element. The plug provides resilient buffer for impact force on the resilient plug by the core-element. The plugged end is faced down to the bottom of the dispenser and bottom of the vial is faced up when the vial is placed into the dispenser. The height of the vial is made longer than the diameter of the main chamber. After placed inside the dispenser, the vial is kept in upside-down position and is impossible to tip over into a reverse direction.

A still a further form of the invention, the secondary compartment having second reactive component and a core-element is a glass vial having an open end having a screw-thread neck socket. The side wall of the glass vial is made thicker to resistant the accident fall with side collisions. The open end can be caped by a resilient gasket. The resilient gasket can be caped on the neck with a screw cap caped on the screw-thread neck socket.

Under typical assembly of the vial, first, the core-element is inserted in the vial, second, a precisely measured volume of second reactive component is filled in the vial, the third, the resilient gasket is caped on the open end of vial with a screw cap caped on the screw-thread neck socket.

Under other typical assembly of the vial, first, the core-element is inserted in the vial, second, the system is evacuated under vacuum, the third, precisely measured volume of second reactive component is filled in the vial under vacuum, the forth, the resilient gasket is caped on the open end of vial with a screw cap caped on the screw-thread neck socket.

Other procedure is practiced. First, the core-element is inserted in the vial, second, a precisely measured volume of second reactive component is filled in the vial, the third, the system is evacuated under vacuum, the forth, the resilient gasket is caped on the open end of vial with a screw cap caped on the screw-thread neck socket.

Still a procedure is practiced. First, the core-element is inserted in the vial, second, the resilient gasket is caped on the open end of vial with a screw cap caped on the screw-thread neck socket, third the vial is vacuumed by a inserted pipe dipping inside of vial, and finally, a precisely measured volume of second reactant component is filled in the vial.

The resilient gasket seals the second reactive component and the core-element. The gasket provides resilient buffer for impact force on the resilient gasket by the core-element. The caped end is faced down to the bottom of the dispenser and bottom of the vial is faced up when the vial is placed into the dispenser. The height of the vial is made longer than the diameter of the main chamber. After placed inside the dispenser the vial is kept in upside-down position and is impossible to tip over into a reverse direction.

The core-element that is enclosed inside the vial is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small vial, and a mixture thereof.

The core-element that is enclosed inside the vial can be selected from a small vial with same design but with smaller dimension, shorter height, and thicker glass thickness than the vial inserted in.

Said the small vial as core-element with same design as the vial inserted in can used to store a third reactive component with smaller volume than the second reactive component.

Said small vial has a plug to seal the third reactive component and the plug is caped with metal cap.

Other design of small vial has a gasket. The gasket is caped with screw cap caped on a screw-thread neck socket of the small vial.

Additional design of small vial has a membrane and the membrane is caped with screw cap caped on screw-thread neck socket on the small vial.

The small vial has thinner glass wall around the neck or bottom or both the neck and the bottom that provides a weak mechanical portion which can be broken by impact when it collides with the vial inserted in by vigorous shaken. The third reactive component is mixed with second reactive component when the neck or the bottom of the small vial is broken by collision with the vial inserted when the dispenser is under vigorous shaken. During further vigorous shaking the broken small vial impacts the accessory vial and caused implosion of the accessory vial. The contents inside of accessory vial is released into the main chamber

In other form this invention is to provide a method for preparation and dispensing of reactive mixture with optimum component ratios containing growing molecule species in a pressure dispenser. Said pressure dispenser comprises a cylinder can made with metal having a concave bottom wall and a vault top that is crimpled on the cylinder. The dispenser is designed for holding at least three reactive components that are loaded with optimum component ratios and cannot generally be mixed together until shortly before use. The pressure dispenser comprises of a main compartment being loaded with the first reactive component with optimum component ratios, and an accessory container, the first accessory container, being loaded with the second reactive component with optimum component ratios and a smaller vial, the secondary accessory container. Said small vial is loaded with third reactive component with optimum component ratios. All three reactive components are precisely measured and consist with optimum component ratios. A liquefied propellant is filled in the main compartment as pressure source.

When impacts are executed by intention with vigorous shaking, the small vial having thinner glass wall around the neck or bottom is broken by impact when it collides with the first accessory container inserted in by vigorous shaken. The third reactive component is mixed with second reactive component when the neck of the small vial is broken by collision with the vial inserted, the first accessory container, when the dispenser is under vigorous shaken. During further vigorous shaking the broken small vial impacts the first accessory container and caused implosion of the first accessory container. After implosion of the first accessory container, both accessory containers are shattered, the mix of all reactive components, including reactants, catalyst, additives, and other inertia ingredients in the multi reactive components stored in the pressure dispenser occurs under additional shaking. A reactive mixture with optimum component ratios containing growing molecule species is prepared following implosion of the first accessory container and mixing of multi reactive components when the dispenser is under vigorously shaking by intention. A valve connecting with a nozzle and having valve housing is mounted in the top that control dispensing. The housing has a dip tube dipping into the bottom of can. A filter is connected at the end of the dip tube. The dispensing valve held shut by the pressure inside of the can and a coil spring against a resilient gasket mounted in the valve housing. When the valve is actuated the reactive mixture containing reactive mixture with optimum component ratios containing growing molecule species is dispensed and exited from the nozzle.

The dispensers are packed and placed upright. The height of the vial is made longer than the diameter of the main chamber. After placed inside the dispenser, the vial is kept in upside-down position and is impossible to tip over into a reverse direction. When the resilient plug is caped with a thin metal cap on vial neck, each core-element in dispenser has been rested on the resilient plug. When the resilient plug is caped with a screw cap caped on vial screw-thread socket, each core-element in dispenser has been rested on the resilient plug. The first reactive component and the vial are loaded in dispenser main chamber. The plug on the vial seals the second reactive component and the core-element. The plug provides resilient buffer for impact force by the core-element. The vial having the core-element is partial full with secondary reactive component that is in a liquid phase. An empty portion in vial space provides buoyancy in the liquid of the first reactive components. The buoyancy keeps the vial with upside-down position on the bottom of the dispenser regardless the carrier's movements or accidental fall of the package when the dispenser have been packed and stored upright. Impacts by the core-element are unable to open the caped plug on the vial. The resilient plug on the open end of the vial is served as a buffer for safeguarding the core-element impacts during transportation and accidental fall due to the resiliency of the plug.

When resilient gasket is caped on vial screw-thread socket and the core-element has been rested on the resilient gasket. The resilient gasket provides resilient buffer for impact force by the core-element. Impacts by the core-element are unable to open the caped resilient gasket on the vial. The resilient gasket on the open end of the vial is served as a buffer for safeguarding the core-element impacts during transportation and accidental fall due to the resiliency of the resilient gasket.

When resilient membrane is caped on screw-thread socket, the core-element has been rested on the membrane. The membrane provides resilient buffer for impact force by the core-element. Impacts by the core-element are unable to open the caped membrane on the vial. The resilient membrane on the open end of the vial is served as a buffer for safeguarding the core-element impacts during transportation and accidental fall due to the resiliency of the membrane.

The sidewall of the vial is made of thicker glass for an additional safeguard to avoid accidental break of the vial by impact that might activate the admixing of reactive components when the dispenser is sideward fell and collided by side.

An additional safeguard is implemented by a resilient device which has been mounted on the top of the pressure dispenser. The resilient device is comprised of a cap with a resilient material or a resilient annular part of the cap. The resilient device is an additional safeguard for pressure dispenser in the case that impacts occurs when the pressure dispenser is mistakenly packed upside-down during transportation or fall with head of pressure dispenser on the ground.

The accessory container, the vial is made of impact-resistant glass. When the impact force is small, the glass wall of the vial is strong enough to resist the impacts of core-element by unintentional collisions.

The glass thickness of bottom of the vial is designed for broken with vigorous shaking by multiple impacts with the core-element. When the dispenser is shaken back and forth along approximate longitudinal direction of the dispenser by intention, the maximum movement distance of the core-element in the vial can reach the amount of the height of the dispenser minus the thicknesses of vial bottom and the height of the plug. The impact force is equal to the mass of the core-element multiplies the acceleration of the traveling core-element. If the dispenser is vigorously shaken back and forth along longitudinal direction of the dispenser, the impacts of the core-element on vial bottom can make micro cracks in the glass wall. The wall of vial is under the stress of pressure differences of propellant pressure externally and the internal pressure of the vial. Since the pressure of propellant is much high than the pressure of accessory container of second reactive component. The vial wall is under net external stress. Under multiple vigorous impacts the micro cracks grow and the vial is imploded by the implosion under the propellant pressure in the dispenser. When vial is imploded and lass wall is shattered. There is no any wall of accessory container wall exist after implosion. As result, the reactive component which had kept inside of the vial is completely released into the main chamber. This release is complete, since the original seal, the vial wall is no longer exists. All reactive components are mixed and reacted to from the reactive mixture with optimum component ratios containing growing molecule species in the primary chamber.

The implosion of accessory container, the vial, is the main feature that distinguishes with previous inventions. The advantage of the implosion is the completeness of the release of the reactive component which was originally stored inside the accessory container. The completeness of the release provides the completeness of the mixing of all reactive components. No previous art provide such advantage.

A further advantage is the convenience of the activation of the implosion. Vigorous hand shaking easily activates the impacts, and the implosion of the accessory container.

A still further advantage is the security of the integrality of the accessory container that guarantees the safeguard against the transportation and accidental collisions.

A filter is attached in the end of the dip tube. The shattered glass and particles are filtered from the liquid that enters in passage through filter to avoid blocking the passage.

When the valve is actuated by applied force with depressed nozzle, the passage is opened. The propellant pressure pushes the mixture of propellant and reactive mixture to flow through the passage. The mixture passes through the first, the filter, the second, the dip tube, than valve housing, valve, tubular nozzle stem, and final, of the nozzle spout.

Present invention is related to a method for preparing and dispensing a reactive mixture containing at least a growing molecule species by a pressure dispenser, wherein said reactive mixture results from mixing and reacting with the first reactive component and at least one second reactive component, each loaded with precise ratios and maintained separately from one another within a main chamber, and at least one accessory container with a safeguard feature which prevents the mixing of the said reactive components until activation, which is induced by the implosion of said accessory chamber upon impact with a core-element, resulting from vigorous shaking, which aforementioned said reactive mixture is dispensed, and is comprised of any combination of:

(a) A cylindrical can made of metal as said main chamber with a predetermined diameter having a closed bottom and a open top;

(b) A vial made of glass as said accessory container having an open top end with a neck and a bottom closed end, said vial having a height longer than said diameter of said cylindrical can;

(c) A core-element being included inside said vial is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small vial, and a mixture thereof;

(d) A second reactive component being loaded in said vial and contributing vapor pressure upon said vial;

(e) A resilient plug as said safeguard feature, plugging and sealing said top open end of said vial and providing a resilient buffer for impacts excised on said top open end of said vial;.

(f) A thin metal cap capping over said resilient plug and keeping said resilient plug over said top open end and holding onto said neck of said vial;

(g) The vial being placed inside said main chamber with said thin metal cap facing said closed bottom of said cylinder can;

(h) The first reactive component being loaded in said main chamber and contributing vapor pressure upon main chamber;

(i) A mounting cup having a pedestal for a valve housing with a center opening at pedestal, and said mounting cup is hermetically crimped in and sealed in said open top of said cylindrical can;

(j) A valve housing having an open end and having base with a hollow nipple receiving a dip tube that extending downward, a filter is mounted to the end of dip tube and approaching to said closed bottom of said cylindrical can for blocking any particles or fragments from flowing into said dip tube, an encircling resilient gasket with a center hole is mounted beneath said pedestal of said mounting cup and attached directly below said center opening at pedestal of said mounting cup, said valve housing is sealed with said open end of said valve housing against said encircling resilient gasket;

(k) A normally shut-off combined fill and discharge slide valve means having a valve stem, said valve stem having an enlarged shoulder, a tubular upper portion with a elongated hole terminated at said enlarged shoulder, at least a stem orifice being perpendicularly bored into said elongated hole of said tubular upper porting and located above said enlarged shoulder; said enlarged shoulder having sealing face on the upper side, said valve stem having a lower base receiving a coil spring compressed between said lower base on one end and against said base of a valve housing on the other end, thereby said valve means is held shut with sealing face on upper side of enlarged shoulder being pushed against said encircling resilient gasket by the force of said compressed coil spring, and a dispensing nozzle with tubular stem connected with the top of tubular upper portion of valve stem;

(l) At least one liquefied propellant being loaded in said main chamber contributing propellant vapor pressure into total pressure of said main chamber, and said total pressure being equal to the sum of propellant vapor pressure and vapor pressure of said first reactive component in said main chamber, When said pressure dispenser having been shaken back and forth vigorously generally in the direction of the longitudinal axis of said pressure dispenser, said core-element in response to the given movement changes of said core-element, generates an impact force being equal to the product of the mass of said core-element multiplied by the acceleration whereby impinging upon said bottom closed end of said vial, therewith under multiple impinging cracks being generated and enlarged in said vial resulting in said implosion of said vial under the pressure difference of said total pressure in said main chamber minus the internal pressure of said glass vial which results in said implosion of said vial thereby causing said first reactive component and second reactive component to be mixed and to react, thereupon which said growing molecule species is produced under an ambient temperature as a result of the mixing and reacting of said first reactive component and second reactive component, wherein a system pressure of said dispenser is equal to total contributions from said propellant and said growing molecule species, and said reactive component; and

Whereby when force being applied on said dispensing nozzle, said coil spring is compressed further thereupon, said dispensing nozzle with valve stem is thereby depressed down in said valve housing, said sealing face on the upper side of the enlarged shoulder forced leaving said encircling resilient gasket, said stem orifice is uncovered, and said valve means is opened, therein a passage is opened, resulting in said reactive mixture containing growing molecule species and said propellant being conveyed through said passage and passing up said filter, said dip tube, said valve housing, said stem orifice, said upper tubular stem, and said dispensing nozzle during a dispensing operation and causes said dispensing of said mixture from said dispenser under said system pressure.

Other version of present invention is related to a method for preparing and dispensing a reactive mixture containing at least a growing molecule species by a pressure dispenser, wherein said reactive mixture results from mixing and reacting with the first reactive component and at least one second reactive component, each loaded with optimum component ratios and maintained separately from one another within a main chamber, and at least one accessory chamber with a safeguard feature which prevents the mixing of the said reactive components until activation, which is induced by the implosion of said accessory chamber upon impact with a core-element, resulting from vigorous shaking, which aforementioned said reactive mixture is dispensed, and is comprised of:

(a) A cylindrical can made of metal as said main chamber with height longer than diameter having a closed bottom and a open top;

(b) A vial made of glass as said accessory chamber having an open top end with a screw socket and a bottom closed end, said vial having a height longer than said diameter of said cylindrical can;

(c) A core-element being included inside said vial is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small vial, and a mixture thereof;

(d) The second reactive component being loaded in said vial and contributing vapor pressure upon said vial;

(e) A resilient gasket as said safeguard feature, sealing said top open end of said vial and providing a resilient buffer for impacts excised on said top open end of said vial;.

(f) A screw cap capping over said resilient gasket and keeping said resilient gasket over said top open end and holding onto said screw socket of said vial;

(g) A vial being placed inside said main chamber with said thin metal cap facing said closed bottom of said cylindrical can;

(h) The first reactive component being loaded in said main chamber and contributing vapor pressure upon main chamber;

(i) A mounting cup having a pedestal for a valve housing with a center opening at pedestal and said mounting cup is hermetically crimped in and sealed in said open top of said cylindrical can;

(j) The valve housing having an open end in one end and having base in other end with a hollow nipple receiving a dip tube that extending downward, a filter is mounted to the end of dip tube and approaching to said closed bottom of said cylindrical can for blocking any particles or fragments from flowing into said dip tube, an encircling resilient gasket with a center hole is mounted beneath said pedestal of said mounting cup and attached directly below said center opening at pedestal of said mounting cup, said valve housing is sealed against said encircling resilient gasket at said open end of said valve housing;

(k) A normally shut-off combined fill and discharge valve means having a blind gear, one side of said blind gear receiving coil spring that is compressed between said blind gear on one end and against said base of said valve housing on other end, other side of blind gear having sealing face and nipple receiving tubular stem of a dispensing nozzle, said sliding gear is held shut with said sealing face of said blind gear against said encircling resilient gasket by the force of compressed coil spring, said tubular stem of nozzle having a open groove cut or at least an orifice being bored into lower side of said tubular stem, said tubular stem of said dispensing nozzle is slide and received on the nipple of slide gear core-element;

(l) At least one liquefied propellant being loaded in said main chamber contributing propellant vapor pressure into total pressure of said main chamber, and said total pressure being equal to the sum of propellant vapor pressure and vapor pressure of said first reactive component in said main chamber; A pressure dispenser for preparing and dispensing a reactive mixture as described above, whereby when said pressure dispenser having been shaken back and forth vigorously generally in direction of the longitudinal axis of said pressure dispenser, said core-element in response to the given movement changes of the pressure dispenser relative to said core-element, generates an impact force being equal to the product of the mass of said core-element multiplied by the acceleration whereby impinging upon said bottom closed end of said vial, therewith under multiple impinging cracks being generated and enlarged in said vial resulting in said implosion of said vial under the pressure difference of said total pressure in said main chamber minus the internal pressure of said glass vial which results in said implosion of said vial thereby causing said first reactive component and second reactive component to be mixed and to react, thereupon which said growing molecule species are produced under an ambient temperature as a result of the mixing and reacting of said first reactive component and second reactive component, wherein a system pressure of said dispenser is equal to total contributions from said propellant and said growing molecule species, and reactive components.

Whereby when said dispensing nozzle being pressed, said blind gear is thereby forced down in said valve housing, and said sealing face on the upper side of the blind gear leaves said encircling resilient gasket and said coil spring is compressed further thereupon said cut groove or orifice is uncovered and said valve means is opened, therein a passage is opened, resulting in said reactive mixture containing growing molecule species and said propellant being conveyed through said passage and passing up said filter, said dip tube, said valve housing, said blind gear, said open cut groove or orifice, and said dispensing nozzle during a dispensing operation and causes said dispensing of said mixture from said dispenser under said system pressure.

A still other version of present invention is a method for preparing and dispensing a reactive mixture including at least a growing molecule species, wherein said reactive mixture results from mixing and reacting between one first reactive component and at least one second reactive component, each loaded with a precise ratio and maintained separately within a pressure dispenser having a main chamber, and at least one accessory container with a safeguard feature which prevents said mixing of said first reactive component and said second reactive component until an implosion of said accessory container, said method includes:

(a) Providing said second reactive component, said second reactive component is loaded in said accessory container loaded with a core element, said accessory container is sealed with a resilient plug as said safeguard feature; said accessory container is placed in said pressure dispenser with an upside-down orientation;

(b) Providing said first reactive component, said first reactive component is loaded in said main chamber of said pressure dispenser, said pressure dispenser is hermetically crimped with a mounting cup, said mounting cup having a pedestal mounted with a combined filling and discharging valve means;

(c) Providing a propellant, said propellant is filled in said pressure dispenser, said propellant providing pressure source for said pressure dispenser and stress on said accessory container;

(e) Providing reactive mixture by vigorous shaking of said pressure dispenser, said vigorous shaking activates said implosion of said accessory container by impacts with said core-element under said stress, said implosion and said vigorous shaking causes said mixing and said reacting of said first reactive component and said second reactive component, and thereby forming said reactive mixture, and

(f) Dispensing said reactive mixture by applying a force to open said valve means, a mixture of said propellant and said reactive mixture including growing molecule species is dispensed from said pressure dispenser under said pressure source. In this invention, wherein said main chamber of said pressure dispenser is a cylindrical can having a closed bottom wall and an open top, said main chamber has a predetermined diameter.

In this method, wherein said accessory container is a glass vial having a top open end with a neck and a bottom closed end, said glass vial has a height longer than said diameter of said main chamber, said second reactive component and said core-element are loaded in said glass vial, said top open end of said glass vial is plugged with said resilient plug as said safeguard feature, a thin metal cap capping said resilient plug over said top open end of said glass vial and holding onto said neck of said glass vial, and said glass vial is placed inside said main chamber with said thin metal cap facing said closed bottom of said pressure dispenser.

In this method, wherein said accessory container is a glass vial having a top open end with a screw-thread socket and a bottom closed end, said glass vial has a height longer than said diameter of said main chamber, said second reactive component and said core-element are loaded in said glass vial, said top open end of said glass vial is plugged with a resilient plug as said safeguard feature, a screw-thread cap capping said resilient plug over said top open end of said glass vial and holding onto said screw-thread socket of said glass vial, and said glass vial is placed inside said main chamber with said screw-thread cap facing said closed bottom of said pressure dispenser.

In this method, wherein said core-element included inside said accessory container is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small vial, and a mixture thereof.

In this method, wherein said filling and discharging valve means includes a encircling gasket, a valve housing, a slide valve core, a coil spring, a dip tube, a filter, and a nozzle, said valve housing including an open end and a base with a hollow nipple receiving said dip tube, said filter is mounted to the end of said dip tube, said encircling gasket sealing between said open end of said valve housing and said pedestal of said mounting cup, said slide valve core has blind gear shape, downside of said slide valve core receiving said coil spring, upside of said slide valve core having an protruded adapt ring receiving a tubular nozzle stem of said nozzle, said slide valve core is held shut with an encircling sealing face on said protruded adapt ring pushed against said encircling gasket by a force of said coil spring compressed between said slide valve core on one end and against said base of said valve housing on the other end, said tubular nozzle stem has a channel at end with at least one open groove cut, an alternative of said open groove cut is at least an open orifice being bored into said tubular nozzle stem, said tubular nozzle stem received on said protruded adapt ring of said slide valve core.

In this method, whereby when said force is applied to open said valve means, said coil spring is compressed further, thereby said nozzle with said tubular nozzle stem, and slide valve core are sliding down, said encircling sealing face on said slide valve core leaves said encircling gasket and said channel on said tubular nozzle stem is uncovered, thereupon a mixture of said propellant and said reactive mixture including said growing molecule species is conveyed through said filter, said dip tube, said valve housing, said tubular nozzle stem, and exit from said nozzle under said pressure source.

In further another method, wherein said filling and discharging valve means includes a encircling gasket, a valve housing, a slide valve core, a coil spring, a dip tube, a filter, and a nozzle, said valve housing including an open end and a base with a hollow nipple receiving said dip tube, said filter is mounted to the end of said dip tube, said encircling gasket sealing between said open end of said valve housing and said pedestal of said mounting cup, said slide valve core has an enlarged shoulder and an upper portion with a tubular valve stem adapting a tubular nozzle stem of said nozzle, said tubular valve stem has an elongated hole terminated at said enlarged shoulder, at least a stem orifice is bored into said elongated hole of said tubular valve stem and located above said enlarged shoulder as a channel, downside of said slide valve core receiving said coil spring, upside of said slide valve core has a sealing ring on said enlarged shoulder around said tubular valve stem, said slide valve core is held shut with said sealing ring on said enlarged shoulder pushed against said encircling gasket by a force of said coil spring compressed between said slide valve core on one end and against said base of said valve housing on the other end, said tubular nozzle stem is received with said tubular valve stem of said slide valve core.

In further method, whereby when said force is applied to open said valve means, said coil spring is compressed further, thereby said nozzle with said tubular nozzle stem, and slide valve core with tubular valve stem are sliding down, said sealing ring on said enlarged shoulder of said slide valve core leaves said encircling gasket and said channel on said tubular valve stem is uncovered, thereupon a mixture of said propellant and said reactive mixture including said growing molecule species is conveyed through said filter, said dip tube, said valve housing, said tubular valve stem, said tubular nozzle stem, and exit from said nozzle under said pressure source.

In this method, whereby when said pressure dispenser having been shaken back and forth vigorously, said core-element in response to the given movement changes of said core-element, generates an impact force whereby impinging upon said accessory container, therewith under multiple impinging cracks being generated and enlarged in said accessory container resulting in said implosion of said accessory container under said stress with said pressure source results in said implosion of said accessory container thereby causing said second reactive component to be released into said main chamber, whereby said growing molecule species is produced as a result of the mixing and reacting of said first reactive component and second reactive component.

In this method, further including a step following said dispensing said reactive mixture, said step is selected from the group consisting of heating, IR heating, microwave heating, UV irritation, electron beam irritation, grafting reaction, telomerisation reaction, telechelic reaction, and chemical modification.

In this invention further includes a method for preparing and dispensing a reactive mixture including at least a growing molecule species, wherein said reactive mixture results from mixing and reacting between one first reactive component, one second reactive component, and one third reactive component, each loaded with a precise ratio and maintained separately within a pressure dispenser having a main chamber, and at least an accessory vial, and a main vial with a safeguard feature which prevents said mixing of the reactive components until an implosion of said main vial and broken of said accessory vial, said method includes:

(a) Providing said third reactive component, said third reactive component is loaded in said accessory vial, said accessory vial is contained in said main vial;

(b) Providing said second reactive component, said second reactive component is loaded in said main vial contained with said accessory vial, said main vial is sealed with a resilient plug as said safeguard feature; said main vial is placed in said pressure dispenser with an upside-down orientation;

(c) Providing said first reactive component, said first reactive component is loaded in said main chamber of said pressure dispenser, said pressure dispenser is hermetically crimped with a mounting cup, said mounting cup having a pedestal mounted with a combined filling and discharging valve means having a dip tube, and a filter;

(d) Providing a propellant, said propellant is filled in said pressure dispenser, said propellant providing pressure source for said pressure dispenser and stress on said main vial;

(e) Providing reactive mixture by vigorous shaking of said pressure dispenser, said vigorous shaking activates said broken of said accessory vial by impacts with main vial and activates said implosion of said main vial by impacts with said broken accessory vial under said stress, said implosion and said vigorous shaking causes said mixing and said reacting of said first reactive component, said second reactive component, and said third reactive component, and thereby forming said reactive mixture, and

(f) Dispensing said reactive mixture by applying a force to open said valve means, a mixture of said propellant and said reactive mixture including growing molecule species is dispensed from said pressure dispenser under said pressure source.

In this invention also includes a method for preparing and dispensing a reactive mixture including at least a growing molecule species, wherein said reactive mixture results from mixing and reacting between one first reactive component, one second reactive component, and one third reactive component, each loaded with a precise ratio and maintained separately within a pressure dispenser having a main chamber, and at least one first vial with safeguard feature, and one second vial with a safeguard feature which prevent said mixing until implosion of said first vial and implosion of said second vial, said method includes:

(a) Providing said second reactive component, said second reactive component is loaded in said first vial loaded with a first core-element, said first vial is sealed with a resilient plug as said safeguard feature; said first vial is placed in said pressure dispenser with an upside-down orientation;

(b) Providing said third reactive component, said third reactive component is loaded in said second vial loaded with a second core-element, said second vial is sealed with a resilient plug as said safeguard feature; said second vial is placed in said pressure dispenser with an upside-down orientation;

(c) Providing said first reactive component, said first reactive component is loaded in said main chamber of said pressure dispenser, said pressure dispenser is hermetically crimped with a mounting cup, said mounting cup having a pedestal mounted with a combined filling and discharging valve means having dip tube and a filter;

(d) Providing a propellant, said propellant is filled in said pressure dispenser, said propellant providing pressure source for said pressure dispenser and stress on said first vial and said second vial;

(e) Providing reactive mixture by vigorous shaking of said pressure dispenser, said vigorous shaking activates said implosion of said first vial by impacts with said first core-element included in said first vial under said stress, and said implosion of second vial by impacts with said second core-element included in said second vial under said stress, said implosions and said vigorous shaking causes said mixing and said reacting of said first reactive component, said second reactive component, and said third reactive component and thereby forming said reactive mixture, and

(f) Dispensing said reactive mixture by applying a force to open said valve means, a mixture of said propellant and said reactive mixture including growing molecule species is dispensed from said pressure dispenser under said pressure source.

### DESCRIPTION OF PREFERRED EMBODIMENTS

Present invention is related to a method for preparing and dispensing a reactive mixture 300 containing at least a growing molecule species by a pressure dispenser 102, wherein said reactive mixture results from mixing and reacting with the first reactive component (100, 101) and at least one second reactive component (200, 201), each loaded with precise ratios and maintained separately from one another within a main chamber of pressure dispenser 102, and at least one accessory container 210 with a safeguard feature 220 which prevents the mixing of the said reactive components (100, 101 and 200, 201) until activation, which is induced by the implosion of said accessory container 210 upon impact with a core-element 240, resulting from vigorous shaking, which aforementioned said reactive mixture 300 is prepared.

For the purpose of safety and guarantee of pressure resistance, the dispenser 102 has cylindrical shape walls with height longer than diameter. A vault top and concave bottom 105 provide pressure resistant advantage. A vault bottom also resists pressure, but it requires extra part for stand on shelf. Corrosion resistant metal and alloy, such as steel, aluminum, steel tin-plated is the material choice for pressure dispenser 102.

Referring to FIG. 1, the pressure dispenser 102 is a metal or metal alloy cylindrical can. The main chamber of pressure dispenser 102 has a concave bottom wall 105 and an open top 108.

A vial 210 made of glass as the accessory container has an open top end with a neck 212 and a bottom closed end. The vial 210 has a height longer than the diameter of the cylindrical can of pressure dispenser 102.

A core-element 240 is included inside said vial 210. The core-element 240 is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small vial, and a mixture thereof.

The second reactive component 200 with precise amount is loaded in said glass vial 210.

A resilient plug 220 is the safeguard feature for the operation of pressure dispenser. The resilient plug 220 plugs the top open end of the vial 210 and seals the reactive component 200 in the glass vial 210.

The material making the plug 220 and material making the core-element 240 must be chemical and corrosion resistant for the reactive component 200 stored in the glass vial 210.

A thin metal cap 230 is capped over said resilient plug 220. It keeps the resilient plug over the top open end and holds the resilient plug onto the neck 212 of the vial 210.

The loaded vial 210 is placed inside said main chamber of pressure dispenser 102 with the thin metal cap 230 facing the closed bottom 105 of pressure dispenser 102.

The first reactive component with precise amount 100 is loaded in the main chamber of the pressure dispenser 102.

A mounting cup 104 has a pedestal 106 for a valve housing 122 with a center opening at pedestal 106.

For provide corrosion resistant against the mixture 100 of the first reactive component and propellant, the internal surfaces of pressure dispenser 102, the internal surface of mounting cup 104, and metal cap 230 must coated with chemical resistant polymer. The coated layers of cross-linked polymer on the internal surfaces of metal pressure dispenser 102 and the metal cap 230 provide corrosion and solvent resistance against the mixture 100 of the first reactive component and propellant.

A combined fill and discharge valve means mounted in the valve housing 122. The valve housing 122 has an open end 123 and a base with a hollow nipple 126.

An encircling resilient gasket 120 seals between the open end 123 of valve housing 122 and pedestal 106 of the mounting cup 104.

The hollow nipple 126 receives a dip tube 140.

A filter 142 is mounted to the end of dip tube 140.

A normally shut-off combined fill and discharge valve means has a slide valve core 124 with blind gear shape. The internal wall of valve housing 122 provides the sliding guide for slide valve core. The gap 127 between concave gears of slide valve core 124 provides the passage for liquid flow when valve is in opening position.

The down side of slide valve core receives a coil spring 128. The up side of slide valve core has a protruded adapt ring 121 that receives tubular stem 166 of a dispensing nozzle 160. The protruded adapt ring 121 has sealing face 125 seals fluid flow when it contacts against the encircling resilient gasket 120. The slide valve core 124 is held shut with the sealing face 125 on the protruded adapt ring 121 of slide valve core 124 against the encircling resilient gasket 120 by the combined forces of the force of repellant and the force of the coil spring 128 compressed between said slide valve core 124 on one end and against said base of said valve housing 122 on the other end.

The tubular stem 166 of nozzle 160 has a channel 164 close to the end of the tubular stem with open groove cut. An alternative for the open groove cut is at least an open orifice being bored into said tubular stem 166. The tubular stem 166 of the dispensing nozzle 160 is received on the protruded adapt ring 121 of slide valve core 124.

The mounting cup 104 is hermetically crimped in and sealed in said open top 108 of said cylindrical can of pressure dispenser 102.

At least one liquefied propellant is loaded in said main chamber of pressure dispenser 102 that contributing propellant vapor pressure into system pressure. The propellant is filled in the dispenser 102 through the combined fill and discharge valve through the slide valve core 124 mounted in the valve housing 122 with a specially designed propellant filling machine.

All materials, the first reactive component 100 and 101, the second reactive component 200 and 201, and the propellant are loaded with high precision by liquid and propellant filling machines having precise volume measurement, constant temperature control, and volume calibration and adjustment to offset the thermal expansions of liquid materials.

The optimum component ratios are the reactive components ratio for complete reactions of reactive groups or reactive sites with an optimized reaction rate under the application temperature for achieving optimum properties of final product which is formed after dispensing of reactive mixture on the target subjects. The formation of a final product requires a reaction time that depend the reaction kinetics. At ambient temperature, the time for completing chemical reactions to form a final product requires from few minutes to few hours after dispersion.

The propellant may be selected from liquefied organic compounds, hydrocarbons, propane, butane, isobutene, dimethyl ether, 1,1,1,2-tetrafluoroethane, carbon dioxide, ammonia, and a mixture thereof.

Under typical assembly of the vial 210, first, the core-element 240 is inserted in the vial 210, second, a precisely measured volume of second reactive component 200 is loaded in the vial, the third, the resilient plug 220 is plugged on the open end of vial, and finally, the plug is caped on the neck 212 of the vial 210 with a metal cap 230.

Under other typical assembly of the vial 210, first, the core-element 240 is inserted in the vial 210, second, a precisely measured volume of second reactive component 200 is loaded in the vial under vacuum, the third, the resilient plug 220 is plugged on the open end of vial under vacuum, and finally, the plug is caped on the neck 212 of the vial 210 with a metal cap 230.

An additional safeguard is implemented by a dispenser cup which has been mounted on the top of the pressure dispenser 102 but not shown. The dispenser cup is comprised of a cup with a resilient material or a resilient annular part of the cup. The resilient cup-shaped cover is an additional safeguard for pressure dispenser in the case that impacts occurs when the pressure dispenser is packed upside-down during transportation or fall with head of pressure dispenser on the ground.

Under typical assembly of the pressure dispenser, first, the assembled vial is placed in the cylindrical can with the metal cap 230 facing down the bottom 105 of the cylindrical can. Second, a precisely measured volume of the first reactive component 100 is loaded in the cylindrical can. The third, the assembly of mounting cup 104 with the filling and discharge valve means including encircling resilient gasket 120, valve housing 122, slide valve core 124, coil spring 128, the dip tube 140, and the filter 142. Said mounting cup is hermetically crimped in and sealed in the open top 108 of the cylindrical can of pressure dispenser 102. The forth, the repellant is filled in the pressure dispenser 102. The nozzle 160 is assembled in the valve housing with the tubular stem 166 of the dispensing nozzle 160 slide in the protruded adapt ring 121 of slide valve core 124. Finally, the dispenser cup is placed over the nozzle and fitted on the top of the pressure dispenser.

When pressure dispensers 102 are packed in the packages, the dispenser 102 is placed upright. Since the vial 210 is placed in the dispenser 102 with the metal cap facing down to the bottom 105 of the dispenser 102, and the height of the vial 210 is longer than the diameter of the dispenser 102, the orientation of the vial 210 inside of the dispenser cannot change, as the result, the core-element 240 inside of vial 210 has been rested on the resilient plug 220 caped with a thin metal cap 230. The plug on the vial seals the second reactive component 200 and the core-element and provides resilient buffer for impact force by the core-element. The vial 210 having the core-element 240 is partial full with secondary reactive component 200 that is in a liquid phase. An empty portion in vial space 201 provides buoyancy in the liquid of the first reactive component 100. The buoyancy keeps the vial 210 with upside-down position on the bottom of the dispenser 102 regardless the carrier's movements or accidental fall of the package when the dispenser have been packed and stored upright. Impacts by the core-element are unable to open the caped plug 220 on the vial 210. The resilient plug 220 on the open end of the vial 210 is served as a buffer for safeguarding the core-element 240 impacts during transportation and accidental fall due to the resiliency of the plug.

The sidewall of the vial 210 is made of thicker glass for an additional safeguard to avoid accidental break of the vial by impact that might activate the admixing of reactive components when the dispenser is sideward fell and collided by side.

The accessory container, the vial is made of impact-resistant glass. When the momentum of an impact is small, the glass wall of the vial is strong enough for resisting the core-element impacts by unintentional collisions.

FIG. 2 shows the preparation of the reactive mixture containing growing molecular species. When the pressure dispenser is shaken back and forth approximately along longitudinal direction of the dispenser by intention, the maximum movement distance of the core-element 240 in the vial can reach the amount of the height of the dispenser minus the thicknesses of vial bottom and the height of the resilient plug 220. The impact force is equal to the mass of the core-element 240 multiplies the acceleration of the core-element 240. When the core-element changes the direction by impact, the acceleration and the force reaches the maximum. If the dispenser is vigorously shaken back and forth along longitudinal direction of the dispenser 102, the impacts of the core-element on vial bottom can make micro cracks in the glass wall.

The wall of vial is under the stress of pressure differences of propellant pressure externally and the vapor pressure of second reactive component internally (solvent provides the major vapor pressure). Since the pressure of propellant is much high than the pressure of second reactive component. The vial wall is under net external stress.

The glass thickness of bottom of the vial is designed for broken with vigorous shaking by multiple impacts with the core-element 240. Under multiple vigorous impacts the micro cracks grow and the vial is imploded by the implosion under the propellant pressure in the dispenser.

When vial is imploded and glass wall is shattered as shown in the FIG. 2, the second reactive component which had kept inside of the vial is released into the main chamber. All reactive components are mixed and reacted to from the reactive mixture with optimum component ratios containing growing molecule species in the main chamber.

FIG. 3 shows the dispensing of the reactive mixture containing growing molecular species. After implosion of the glass vial, mixing and reacting of reactive components, the reactive mixture is prepared and ready to be applied as shown in FIG. 2.

The port life of the reactive mixtures is designed by the balance the application convenience and the effectiveness of the reactive mixture. During the port life of the reactive mixture, when a force is applied on the dispensing nozzle 160, the coil spring 128 is compressed further, the slide valve core 124 is depressed thereby slide down in valve housing 122 as valve slide guide, and the sealing face 125 on the upper side of the protruded adapt ring 121 leaves the encircling resilient gasket 120, the channel 164 with the open cut groove in tubular nozzle stem 166 is uncovered, and therefore, the valve means is opened. A passage is opened.

The filter 142 is an important element. The shattered glass must be filtered from the liquid that entering the opened passage for preventing particles blocking passage.

When the passage is opened, the mixture 300 of propellant and the reactive mixture containing growing molecule species are conveyed through said passage under propellant pressure. Said mixture 300 is passing up the filter 142, the dip tube 140, the hollow nipple 130, the internal space 129 of valve housing, the space 127 between concave gears of slide valve core 124 and valve housing 122 as slide valve guide, the channel 164 with open cut groove in nozzle stem 166, and the tubular space 168 in the dispensing nozzle 160, and the finally through the spout 162. When dispenser is equipped with fine spout 162, the product is sprayed as fine drops, as soon as the drops leave the spout 162, drops are broken to form aerosol by the evaporation of the propellant during sudden pressure expansion and causes the dispersing of said mixture from the dispenser.

The invention further including at least one step following said dispensing reactive mixture including growing molecule species, said step is selected from the group consisting of heating, IR heating, microwave heating, UV irritation, electron beam irritation, grafting reaction, telomerisation reaction, telechelic reaction, chemical modification, and a mixture thereof.

The invention can be applied for multi-component coating material, multi-component adhesives, multi-component paints, multi-component sealer, multi-component dye, and multi-component dental materials, multi-component operation adhesives, experimental testing materials, multi-component diagnostic materials, multi-component bone restoration materials, multi-component casting and soldering material, multi-component electric and electronic materials, and etc.

This invention relates to a method for preparation and dispensing of reactive mixture with optimum component ratios containing growing molecule species in a pressure dispenser, wherein said growing molecule species containing at least a free hydroxy functional group, said first reactive component includes at least a macromolecule with at least two hydroxy functional groups, and said second reactive component includes at least an isocyanate with at least two isocyanato functional groups.

This invention relates to a method for preparation and dispensing of reactive mixture with optimum component ratios containing growing molecule species in a pressure dispenser, wherein said growing molecule species containing at least a free amino functional group, said first reactive component includes at least a macromolecule with at least two amino functional groups, and said second reactive component includes at least an isocyanate with at least two isocyanato functional groups.

This invention relates to a method for preparation and dispensing of reactive mixture with optimum component ratios containing growing molecule species in a pressure dispenser, wherein said growing molecule species containing at least a free epoxy functional group, said first reactive component includes at least an epoxy oligomer with at least two epoxy functional groups, and said second reactive component includes at least a curing agent, said curing agent is selected from the group consisting of amine, polyamide, anhydride, Lewis acid, urea, melamine, imidazole, BF, amine complex, imide, and a mixture thereof.

This invention relates to a method for preparation and dispensing of reactive mixture with optimum component ratios containing growing molecule species in a pressure dispenser, wherein said growing molecule species containing at least a free carbon-carbon double bond, said first reactive component includes at least an acrylic molecule containing at least a carbon-carbon double bond, and said second reactive component includes at least a substance, said substance is selected from the group consisting of organic peroxide, inorganic peroxide, azo compound, metal alkyl, metathesis catalyst, Bronsted acid, Lewis acid, anionic catalyst, Zeigler-Natta coordination catalyst, organo-metallic compound, metal complex, and a mixture thereof.

This invention relates to a method for preparation and dispensing of reactive mixture with optimum component ratios containing growing molecule species in a pressure dispenser, wherein said growing molecule species containing at least a free carbon-carbon double bond, said first reactive component includes at least an cyclic olefin, said cyclic olefin is selected from the group consisting of monocyclic olefin, bicyclic olefin, polycyclic olefin, cyclic olefin with ester group, cyclic olefin with nitrile group, cyclic olefin with halogen group, oxygen-containing heterocyclic olefin, nitrogen-containing heterocyclic olefin, silicon-containing heterocyclic olefin and a mixture thereof, and said second reactive component includes at least a substance, said substance is selected from the group consisting of metathesis catalyst, Bronsted acid, Lewis acid, anionic catalyst, Zeigler-Natta coordination catalyst, organo-metallic compound, metal alkyl, metal complex, inorganic peroxide, organic peroxide, azo compound, and a mixture thereof.

This invention relates to a method for preparation and dispensing of reactive mixture with optimum component ratios containing growing molecule species in a pressure dispenser, wherein said growing molecular species containing at least a free thio functional group, said first reactive component includes at least a polysulfide oligomer, and second reactive component includes at least a curing agent, said curing agent is selected from the group consisting of lead dioxide, activated manganese dioxide, calcium peroxide, cumene hydroperoxide, alkaline dichromate, p-quinonedioxime, furfurol, dichlorodiphenol, tine oxide, hydrazine, peperidine, magnesium oxide, sulfoxide, epoxy oligomer, isocyanate, potassium permanganate, zinc oxide, and a mixture thereof.

This invention relates to a method for preparation and dispensing of reactive mixture with optimum component ratios containing growing molecule species in a pressure dispenser, wherein said growing molecule species includes at least a free amino-acid group, said first reactive component includes at least a fibrinogen, and said second reactive component includes at least collagen aggregation enzyme.

This invention relates to a method for preparation and dispensing of reactive mixture with optimum component ratios containing growing molecule species in a pressure dispenser, wherein said growing molecular species including at least an imino functional group, said first reactive component includes at least a phenyldiamine, and second reactive component includes at least a dilute solution of hydrogen peroxide.

This invention relates to a method for preparation and dispensing of reactive mixture with optimum component ratios containing growing molecule species in a pressure dispenser, wherein said growing molecular species including at least an conjugated double bond chromophore functional group, said first reactive component includes at least a dye certified for foods, drugs, and cosmetics, said dye certified for foods, drugs, and cosmetics is selected from the groups consisting of azo dye, diazo dye, cyanine dye, rhodamine dye, xanthere dye, fluorine dye, anthraquinone dye, triphenylmethane dye, indole dye, indoline dye, chromoionophore, fluoroionophore, melanin dye, and a mixture thereof, and second reactive component includes at least an agent with a functional group, said functional group is selected from the group consisting of thio, thioacetyl, thiobenzoyl, thiocarbamoyl, thiocarbazono, thiocarbodiazono, thiocarbonohydrazido, thiocabonyl, thiocarboxy, thiocyanato, thioformyl, thionoyl, thioreido, thioxo, mercapto, methionyl, acetylcysteine, cysteine, cysteino, cystine, cystino, cysteino, cystamino, epidithio, epithio, isothiocyanato, thioglycolate, thiolacetate, thioglycolate, thiolactate, thioacetyl, thiobenzoyl, thiocarbamoyl, thiocarbazonol, thiocarbodiazonol, and a mixture thereof.

What is claimed is:

1. A method for preparing and dispensing a reactive mixture including at least a growing molecule species, wherein said reactive mixture results from mixing and reacting between one first reactive component and at least one second reactive component, each loaded with a precise ratio and maintained separately within a pressure dispenser having a main chamber, and at least one accessory container with a safeguard feature which prevents said mixing of said first reactive component and said second reactive component until an implosion of said accessory container, said method includes:
(1a) providing said second reactive component, said second reactive component is loaded in said accessory container loaded with a core element, said accessory container is sealed with a resilient plug as said safeguard feature, said accessory container is placed in said pressure dispenser with an upside-down orientation;
(1b) providing said first reactive component, said first reactive component is loaded in said main chamber of said pressure dispenser, said pressure dispenser is hermetically crimped with a mounting cup, said mounting cup having a pedestal mounted with a valve means for filling and discharging;
(1c) providing a propellant, said propellant is filled in said pressure dispenser through said valve means, said propellant providing pressure source for said pressure dispenser and stress on said accessory container;
(1d) providing reactive mixture by vigorous shaking of said pressure dispenser, said vigorous shaking activates said implosion of said accessory container by impacts with said core-element under said stress, said implosion and said vigorous shaking causes said mixing and said reacting of said first reactive component and said second reactive component, and thereby forming said reactive mixture, and
(1e) dispensing said reactive mixture by applying a force to open said valve means, thereby a mixture of said propellant and said reactive mixture including at least a growing molecule species is dispensed from said pressure dispenser under said pressure source.

2. The method of claim 1, wherein said growing molecule species containing at least a free hydroxy functional group, said first reactive component includes at least a macromolecule with at least two hydroxy functional groups, and said second reactive component includes at least an isocyanate with at least two isocyanato functional groups.

3. The method of claim 1, wherein said growing molecule species containing at least a free amino functional group, said first reactive component includes at least a macromolecule with at least two amino functional groups, and said second reactive component includes at least an isocyanate with at least two isocyanato functional groups.

4. The method of claim 1, wherein said growing molecule species containing at least a free epoxy functional group, said first reactive component includes at least an epoxy oligomer with at least two epoxy functional groups, and said second reactive component includes at least a curing agent, said curing agent is selected from the group consisting of amine, polyamide, anhydride, Lewis acid, urea, melamine, imidazole, BF, amine complex, imide, and a mixture thereof.

5. The method of claim 1, wherein said growing molecule species containing at least a free carbon-carbon double bond, said first reactive component includes at least an acrylic molecule includes at least a carbon-carbon double bond, and said second reactive component includes at least a substance, said substance is selected from the group consisting of organic peroxide, inorganic peroxide, azo compound, metal alkyl, metathesis catalyst, Bronsted acid, Lewis acid, anionic catalyst, Zeigler-Natta coordination catalyst, organo-metallic compound, metal complex, and a mixture thereof.

6. The method of claim 1, wherein said growing molecule species containing at least a free carbon-carbon double bond, said first reactive component includes at least an cyclic olefin, said cyclic olefin is selected from the group consisting of monocyclic olefin, bicyclic olefin, polycyclic olefin, cyclic olefin with ester group, cyclic olefin with nitrile group, cyclic olefin with halogen group, oxygen-containing heterocyclic olefin, nitrogen-containing heterocyclic olefin, silicon-containing heterocyclic olefin and a mixture thereof, and said second reactive component includes at least a substance, said substance is selected from the group consisting of metathesis catalyst, Bronsted acid, Lewis acid, anionic catalyst, Zeigler-Natta coordination catalyst, organo-metallic compound, metal alkyl, metal complex, inorganic peroxide, organic peroxide, azo compound, and a mixture thereof.

7. The method of claim 1, wherein said growing molecular species containing at least a free thio functional group, said first reactive component includes at least a polysulfide oligomer, and second reactive component includes at least a curing agent, said curing agent is selected from the group consisting of lead dioxide, activated manganese dioxide, calcium peroxide, cumene hydroperoxide, alkaline dichromate, p-quinonedioxime, furfurol, dichlorodiphenol, tine oxide, hydrazine, peperidine, magnesium oxide, sulfoxide, epoxy oligomer, isocyanate, potassium permanganate, zinc oxide, and a mixture thereof.

8. The method of claim 1, wherein said growing molecule species includes at least a free amino-acid group, said first reactive component includes at least a fibrinogen, and said second reactive component includes at least collagen aggregation enzyme.

9. The method of claim 1, wherein said growing molecular species including at least an imino functional group, said first reactive component includes at least a phenyldiamine, and second reactive component includes at least a dilute solution of hydrogen peroxide.

10. The method of claim 1, wherein said growing molecular species including at least an conjugated double bond chromophore functional group, said first reactive component includes at least a dye certified for foods, drugs, and cosmetics, said dye certified for foods, drugs, and cosmetics is selected from the groups consisting of azo dye, diazo dye, cyanine dye, rhodamine dye, xanthere dye, fluorine dye, anthraquinone dye, triphenylmethane dye, indole dye, indoline dye, chromoionophore, fluoroionophore, melanin dye, and a mixture thereof, and second reactive component includes at least an agent with a functional group, said functional group is selected from the group consisting of thio, thioacetyl, thiobenzoyl, thiocarbamoyl, thiocarbazono, thiocarbodiazono, thiocarbonohydrazido, thiocabonyl, thiocarboxy, thiocyanato, thioformyl, thionoyl, thioreido, thioxo, mercapto, methionyl, acetylcysteine, cysteine, cysteino, cystine, cystino, cysteino, cystamino, epidithio, epithio, isothiocyanato, thioglycolate, thiolacetate, thioglycolate, thiolactate, thioacetyl, thiobenzoyl, thiocarbamoyl, thiocarbazonol, thiocarbodiazonol, and a mixture thereof.

11. The method of claim 1, wherein said main chamber of said pressure dispenser is a cylindrical can having a closed bottom wall and an open top, said main chamber has a predetermined diameter.

12. The method of claim 1, wherein said accessory container is a glass vial having a top open end with a neck and a bottom closed end, said glass vial has a height longer than a diameter of said main chamber, said second reactive component and said core-element are loaded in said glass vial, said top open end of said glass vial is plugged with said resilient plug as said safeguard feature, a thin metal cap capping said resilient plug over said top open end of said glass vial and holding onto said neck of said glass vial, and said glass vial is placed inside said main chamber with said thin metal cap facing said closed bottom of said pressure dispenser.

13. The method of claim 1, wherein said accessory container is a glass vial having a top open end with a screw-thread socket and a bottom closed end, said glass vial has a height longer than a diameter of said main chamber, said second reactive component and said core-element are loaded in said glass vial, said top open end of said glass vial is plugged with a resilient plug as said safeguard feature, a screw-thread cap capping said resilient plug over said top open end of said glass vial and holding onto said screw-thread socket of said glass vial, and said glass vial is placed inside said main chamber with said screw-thread cap facing said closed bottom of said pressure dispenser.

14. The method of claim 1, wherein said core-element included inside said accessory container is selected from the group consisting of a metal sphere, a metal ellipsoid, a metal cylinder, a metal elliptic cylinder, a metal gear, a glass sphere, a glass ellipsoid, a glass cylinder, a glass elliptic cylinder, a glass gear, a glass capillary closed in both end, a small glass vial, and a mixture thereof.

15. The method of claim 1, whereby when said pressure dispenser having been shaken back and forth vigorously, said core-element in response to the given movement changes of said core-element, generates an impact force whereby impinging upon said accessory container, therewith under multiple impinging cracks being generated and enlarged in said accessory container results in said implosion of said accessory container under said stress with said pressure source thereby causing said second reactive component to be released into said main chamber, whereby said growing molecule species is produced as a result of the mixing and reacting of said first reactive component and second reactive component.

16. The method of claim 1, wherein said valve means for filling and discharging includes a encircling gasket, a valve housing, a slide valve core, a coil spring, a dip tube, a filter, and a nozzle, said valve housing including an open end and a base with a hollow nipple receiving said dip tube, said filter is mounted to the end of said dip tube, said encircling gasket sealing between said open end of said valve housing and said pedestal of said mounting cup, said slide valve core has blind gear shape, downside of said slide valve core receiving said coil spring, upside of said slide valve core having an protruded adapt ring receiving a tubular nozzle stem of said nozzle, said slide valve core is held shut with an encircling sealing face on said protruded adapt ring pushed against said encircling gasket by a force of said coil spring compressed between said slide valve core on one end and against said base of said valve housing on the other end, said tubular nozzle stem has a channel at end with at least one open groove cut, an alternative of said open groove cut is at least an open orifice being bored into said tubular nozzle stem, said tubular nozzle stem received on said protruded adapt ring of said slide valve core;
whereby when said force is applied to open said valve means, said coil spring is compressed further, thereby said nozzle with said tubular nozzle stem, and slide valve core are sliding down, said encircling sealing face on said slide valve core leaves said encircling gasket and said channel on said tubular nozzle stem is uncovered, thereupon a mixture of said propellant and said reactive mixture including said growing molecule species is conveyed through said filter, said dip tube, said valve housing, said tubular nozzle stem, and exit from said nozzle under said pressure source.

17. The method of claim 1, wherein said valve means for filling and discharging includes a encircling gasket, a valve housing, a slide valve core, a coil spring, a dip tube, a filter, and a nozzle, said valve housing including an open end and a base with a hollow nipple receiving said dip tube, said filter is mounted to the end of said dip tube, said encircling gasket sealing between said open end of said valve housing and said pedestal of said mounting cup, said slide valve core has an enlarged shoulder and an upper portion with a tubular valve stem adapting a tubular nozzle stem of said nozzle, said tubular valve stem has an elongated hole terminated at said enlarged shoulder, at least a stem orifice is bored into said elongated hole of said tubular valve stem and located above said enlarged shoulder as a channel, downside of said slide valve core receiving said coil spring, upside of said slide valve core has a sealing ring on said enlarged shoulder around said tubular valve stem, said slide valve core is held shut with said sealing ring on said enlarged shoulder pushed against said encircling gasket by a force of said coil spring compressed between said slide valve core on one end and against said base of said valve housing on the other end, said tubular nozzle stem is received with said tubular valve stem of said slide valve core;
whereby when said force is applied to open said valve means, said coil spring is compressed further, thereby said nozzle with said tubular nozzle stem, and slide valve core with tubular valve stem are sliding down, said sealing ring on said enlarged shoulder of said slide valve core leaves said encircling gasket and said channel on said tubular valve stem is uncovered, thereupon a mixture of said propellant and said reactive mixture including said growing molecule species is conveyed through said filter, said dip tube, said valve housing, said tubular valve stem, said tubular nozzle stem, and exit from said nozzle under said pressure source .

18. The method of claim 1, further including at least a step following said dispensing said reactive mixture, said step is selected from the group consisting of heating, IR heating, microwave heating, UV irritation, electron beam irritation, grafting reaction, telomerisation reaction, telechelic reaction, chemical modification, and a mixture thereof.

19. A method for preparing and dispensing a reactive mixture including at least a growing molecule species, wherein said reactive mixture results from mixing and reacting between one first reactive component, one second reactive component, and one third reactive component, each loaded with a precise ratio and maintained separately within a pressure dispenser having a main chamber, and at least an accessory vial, and a main vial with a safeguard feature which prevents said mixing of the reactive components until an implosion of said main vial and broken of said accessory vial, said method includes:
(19a) providing said third reactive component, said third reactive component is loaded in said accessory vial, said accessory vial is sealed and included in said main vial;
(19b) providing said second reactive component, said second reactive component is loaded in said main vial including said accessory vial, said main vial is sealed with a resilient plug as said safeguard feature; said main vial is placed in said pressure dispenser with an upside-down orientation;
(19c) providing said first reactive component, said first reactive component is loaded in said main chamber of said pressure dispenser, said pressure dispenser is hermetically crimped with a mounting cup, said mounting cup having a pedestal mounted with a valve means for filling and discharging having a dip tube, and a filter;
(19d) providing a propellant, said propellant is filled in said pressure dispenser, said propellant providing pressure source for said pressure dispenser and stress on said main vial;
(19e) providing reactive mixture by vigorous shaking of said pressure dispenser, said vigorous shaking activates said broken of said accessory vial by impacts with main vial and activates said implosion of said main vial by impacts with said broken accessory vial under said stress, said implosion and said vigorous shaking causes said mixing and said reacting of said first reactive component, said second reactive component, and said third reactive component, and thereby forming said reactive mixture; and
(19f) dispensing a mixture of said propellant and said reactive mixture including at least a growing molecule species by applying a force to open said valve means under said pressure source.

20. A method for preparing and dispensing a reactive mixture including at least a growing molecule species, wherein said reactive mixture results from mixing and reacting between one first reactive component, one second reactive component, and one third reactive component, each loaded with a precise ratio and maintained separately within a pressure dispenser having a main chamber, and at least one first vial with safeguard feature, and one second vial with a safeguard feature which prevent said mixing until implosion of said first vial and implosion of said second vial, said method includes;
(20a) providing said second reactive component, said second reactive component is loaded in said first vial loaded with a first core-element, said first vial is sealed with a resilient plug as said safeguard feature; said first vial is placed in said pressure dispenser with an upside-down orientation;
(20b) providing said third reactive component, said third reactive component is loaded in said second vial loaded with a second core-element, said second vial is sealed with a resilient plug as said safeguard feature; said second vial is placed in said pressure dispenser with an upside-down orientation;
(20c) providing said first reactive component, said first reactive component is loaded in said main chamber of said pressure dispenser, said pressure dispenser is hermetically crimped with a mounting cup, said mounting cup having a pedestal mounted with a valve means for filling and discharging having dip tube and a filter;
(20d) providing a propellant, said propellant is filled in said pressure dispenser, said propellant providing pressure source for said pressure dispenser and stress on said first vial and said second vial;
(20e) providing reactive mixture by vigorous shaking of said pressure dispenser, said vigorous shaking activates said implosion of said first vial by impacts with said first core-element included in said first vial under said stress, and said implosion of second vial by impacts with said second core-element included in said second vial under said stress, said implosions and said vigorous shaking causes said mixing and said reacting of said first reactive component, said second reactive component, and said third reactive component and thereby forming said reactive mixture; and
(20f) dispensing said reaction mixture by applying a force to open said valve means, a mixture of said propellant and said reactive mixture including at least a growing molecule species is dispensed from said pressure dispenser under said pressure source.
